(19) United States
(12) Patent Application Publication
(10) Pub. No.: US 2022/0392576 A1
(43) Pub. Date: Dec. 8, 2022

(54) METHOD FOR DETECTION AND IDENTIFICATION OF KNOWN AND EMERGENT PATHOGENS

(71) Applicant: Government of the United States as Represented by the Secretary of the Air Force, Wright-Patterson AF, OH (US)

(72) Inventor: James C Baldwin, Huber Heights, OH (US)

(73) Assignee: Government of the United States as Represented by the Secretary of the Air Force, Wright-Patterson AFB, OH (US)

(21) Appl. No.: 17/816,169

(22) Filed: Jul. 29, 2022

Related U.S. Application Data
(62) Division of application No. 15/908,765, filed on Feb. 28, 2018.
(60) Provisional application No. 62/464,604, filed on Feb. 28, 2017.

(51) Int. Cl. G16B 30/10

(52) U.S. Cl. CPC G16B 30/10

(57) ABSTRACT

A method of detecting and identifying pathogens in a sample comprising a plurality of genetic sequences. A plurality of electronic sequence reads corresponding to the plurality of genetic sequences is received and sampled to form a sample set. The sample set is iteratively and electronically compared to a plurality of pathogen sequences to create a detection group, which populates a putative genome data structure. A distance score is measured between each electronic sequence read of the sampled set to each pathogen sequence of the putative genome data structure. A hit score is calculated by comparing the distance score to a threshold value. A plurality of clusters of the electronic sequence reads of the sample set is formed to maximize the cluster hit score and to minimize a difference in distance scores of the cluster. A respective taxonomic group assigned to electronic reads of the sample set after clustering is displayed.

This application is a division of U.S. application Ser. No. 15/908,765 filed Feb. 28, 2018 (pending), which claims the benefit of and prior to, under 35 U.S.C. § 119(e), U.S. Provisional Patent Application No. 62/464,604 filed on Feb. 28, 2017. The entire content of each application is incorporated herein by reference in its entirety.

### RIGHTS OF THE GOVERNMENT

The invention described herein may be manufactured and used by or for the Government of the United States for all governmental purposes without the payment of any royalty.

### FIELD OF THE INVENTION

The present invention relates generally to methods pathogen identification and, more particularly, to methods of detecting and identifying pathogens.

### BACKGROUND OF THE INVENTION

Conventional methods used to detect pathogenic diseases are limited to a small number of potential microbial targets and require foreknowledge of what pathogenic diseases should be logically searched. Once possible pathogenic diseases are determined, developed primers and probes are used in conventional assay methods to identify whether the particular disease is present. However, the foreknowledge of what pathogenic diseases and tests to consider requires a vast amount of manpower and technical resources. An alternative, particularly when unexpected pathogens are present, would be to use a single test for all pathogens; however, impractical with the current state of the art, especially true in resource poor locations or forward deployed troops.

One conventional process, Next Generation Sequencing (“NGS”), has progressed to the point where sequencing can be used to create advanced assays for detecting disease and rapidly emerging infectious diseases based on genetic data. Some of NGS systems can now be deployed to resource-poor locations, such as field labs. However, one barrier to widespread adoption of NGS remains: data analysis. Data analysis remains a manual process and requires highly skilled technicians with significant computational load.

As to computational load, a typical genome class sequence may yield approximately 10 GB of data. The computational load is anticipated to grow with each generation of instrument improvement. Much of this data is redundant and may not be of practical use in pathogen identification, but manual filtration and cleaning of the data can be time consuming and requires significant attention to detail. Again, such activities are conventionally accomplished manually by highly trained personal that must ensure every sample is managed in the same exacting way, without the introduction of human bias. Hence, what is needed is an efficient automated method of identification that requires lower perceived complexity and that will automatically ensure a precise standard of data analysis is met for every sample.

For specific activities, such as pathogen identification, automation and fielding may be achieved without complicated requirements. Fielding may include point-of-care clinical testing sites staffed by personnel having basic health skill sets but lacking the specialized skill set to perform advanced sequencing and/or complex pathogen identification. Other more complex variants of the methods, such as the identification of novel bioengineered threats, may still require special services, off-site. Yet, such a field system could more efficiently use limited resources, for example, by only calling on Internet services when necessary (or available).

There remains a need for a single kit or process, suitable for field use, which can extract DNA and analyze all genetic material in the sample in order to make accurate organism identification without a prior knowledge of the infecting organism.

### SUMMARY OF THE INVENTION

Embodiments of the present invention overcome the foregoing problems and other shortcomings, drawbacks, and challenges of detecting and identifying known and emergent pathogens. While the present invention will be described in connection with certain embodiments, it will be understood that present, the invention is not limited to these exemplified embodiments. To the contrary, the present invention includes all alternatives, modifications, and equivalents as may be included within the spirit and scope of the present invention.

According to one embodiment of the present invention, a method of detecting and identifying pathogens in a sample comprising a plurality of genetic sequences. A plurality of electronic sequence reads corresponding to the plurality of genetic sequences is received and sampled to form a sample set. The sample set is iteratively and electronically compared to a plurality of pathogen sequences to create a detection group, which populates a putative genome data structure. A distance score is measured between each electronic sequence read of the sampled set to each pathogen sequence of the putative genome data structure. A hit score is calculated by comparing the distance score to a threshold value. A plurality of clusters of the electronic sequence reads of the sample set is formed to maximize the cluster hit score and to minimize a difference in distance scores of the cluster. A respective taxonomic group assigned to electronic reads of the sample set after clustering is displayed.

Another embodiment of the present invention includes a computerized system having an electronic filtering subsystem and an electronic mapping subsystem. The electronic filtering subsystem is configured to electronically receive a plurality of electronic sequence reads associated with a sample comprising a respective plurality of genetic sequences, and to electronically sample the plurality of subject electronic sequence reads to define a selected set of sequence reads. The electronic filtering subsystem is also configured to electronically compare the selected set of sequence reads to a plurality of known genetic sequences, and, upon electronically detecting a match between a sequence read of the selected set and at least one known genetic sequence of the plurality, electronically defined as a detection group, electronically populating a putative genome data structure comprising the detection group. The electronic mapping subsystem is configured to electronically compare the sequence reads of the selected set against the known genetic sequences of the putative genome data structure. Upon electronically detecting a match between a sequence read of the selected set and at least one known genetic sequence of the plurality above a match threshold, the electronic mapping subsystem is configured to electronically calculate a distance score defined by a quality match between the sequence read of the selected set and each genetic sequence of the putative genome data structure, and to electronically calculate a hit score from the distance score for each sequence read of the selected set, the hit score being a comparison of the distance score of a respective electronic sequence read to a threshold. The electronic mapping subsystem is also configured to electronically cluster the electronic sequence reads of the selected set according to a respective association of the a taxonomic group, the hit score, and the distance score, and upon electronic detection of satisfaction of the electronic clustering, electronically assigning the electronic sequence reads as belonging to the taxonomic group associated with the detection group.

In one aspect, embodiments of the present invention relate to a computer-implemented method for identifying pathogens in a sample comprising a plurality of subject genetic sequences. In this method, a first plurality of electronic sequence reads associated with the sample may be received. From this first plurality of genetic sequences, a selected set of subject sequence reads may be selected electronically. This selected set of subject sequence reads may be iteratively compared electronically against a second plurality of known genetic sequences to create a detection group, wherein the detection group may include at least one known genetic sequence of the second plurality matched by the selected set. A putative genomic data structure may be populated electronically with the detection group. The first plurality of subject sequence reads may be compared electronically against the putative genomic data structure to define compared subject sequence reads. A respective hit score and a respective distance score may be calculated for each of the compared subject sequence reads relative to the detection group of the putative genomic data structure. Upon detection of a respective hit score and a respective distance score for each of the compared subject sequence reads which exceeds a threshold value, the compared subject sequence read having such a hit score and distance score may be assigned to a taxonomic group associated with the detection group. The respective taxonomic group assigned to each of the compared subject sequence reads having such a hit score and distance score may be displayed.

In this embodiment the step of comparing the first plurality against the putative genomic data structure may further include electronically calculating, for each of the compared subject electronic sequence reads, a respective entropy score. The calculated entropy score of may indicate a direct match to the detection group of the putative genomic data structure. In this embodiment, a calculated entropy score of greater than 1 may indicate an inexact match to the detection group of the putative genomic data structure. Furthermore, the step of comparing electronically the first plurality against the putative genomic data structure may further include determining electronically a respective identity of each of the compared subject sequence reads by comparing the hit scores, distance scores, and entropy scores and displaying electronically the respective identity of each of the compared subject sequence reads.

This embodiment may include selecting the selected set of subject sequence reads and further including electronically reverse mapping the first plurality against a filtered plurality of known genetic sequences prior to selecting the selected set. Also, the filtered plurality may include known human genetic sequences, taxonomic information, or both. Furthermore, the second plurality may include known agents of concern and the sample may be drawn from a test subject to formulate a test group.

In this embodiment, the respective taxonomic group assigned to each of the compared subject sequence reads may be selected from the group consisting of known pathogens and unknown pathogens. Furthermore, the subject sequence reads of the first plurality of step (a) may be characterized by a respective length of at least 75 base pairs. This embodiment may also supplement the step of comparing the first plurality against the putative genomic data structure by electronically matching each compared subject sequence read which fails to exceed the threshold value as belonging to at least one of: a protein sequence, a motif sequence, a toxin-virulent sequence, or a warfare sequence upon electronic detection of the respective hit score and distance score for each of the compared subject electronic sequence reads which fails to exceed the threshold value.

In another embodiment the computerized system may include an electronic filtering subsystem structured to: electronically receive a first plurality of subject electronic sequence reads associated with a sample comprising subject genetic sequences; electronically select a subset of the first plurality to define a selected set of subject sequence reads; electronically compare the selected set to a second plurality of known genetic sequences; and upon electronically detecting satisfaction of a first match threshold between the selected set and at least one of the second plurality of known genetic sequences, defined as a detection group, electronically populate a putative genome data structure comprising the detection group.

This computerized system also may include an electronic mapping subsystem configured to: electronically compare the first plurality against the putative genome data structure by comparing each of the first plurality of subject sequence reads to the detection group of the putative genome data structure; upon electronically detecting satisfaction of a second match threshold between at least one of the first plurality and the detection group, electronically defined as a compared match; electronically populate the putative genome data structure by retrieving a taxonomic group associated with the compared match to electronically calculate a hit score and a distance score for the compared match; electronically recording to the putative genome data structure a respective association of the compared match with the detection group, the taxonomic group, the hit score, and the distance score; using the putative genome data structure, electronically identifying the subject genetic sequences of the sample associated with the first plurality to define identified subject sequence reads, including electronically calculating a respective entropy score for each of the first plurality; and upon electronic detection of satisfaction of a third match threshold among the respective entropy scores for the identified subject sequence reads, electronically assigning the identified subject sequence reads as belonging to the taxonomic group associated with the detection group.

In this embodiment a respective entropy score of 1 may indicate a direct match of the identified subject sequence read to the detection group of the putative genomic data structure. Furthermore, a respective entropy score which is greater than 1 may indicate an inexact match of the identified subject sequence read to the detection group of the putative genomic data structure. This embodiment may include an electronic reporting subsystem configured to electronically display at least one of the respective taxonomic group associated with each of the compared subject sequence reads and the respective taxonomic group assigned to each of the identified subject sequence reads.

This embodiment may also include wherein the filtering subsystem further structured to electronically filter the results against genetic sequence or taxonomic group information to reduce numerosity of the results (signal to noise) of the plurality of subject electronic sequence reads against a filtered genetic sequence. Furthermore, the filtering subsystem may further be structured to electronically filter the results against genetic sequence or taxonomic group information to reduce numerosity of the results (signal to noise) of the plurality of subject electronic sequence reads against a filtered genetic sequence.

This embodiment may include the plurality of known genetic sequences including a known class A pathogen sequence. Furthermore, the plurality of subject genetic sequences may include at least one of a DNA sequence and an RNA sequence. Also, the respective taxonomic group assigned to each of the identified subject sequence reads may be of a type selected from the group consisting of known pathogens and unknown pathogens. Lastly, the identified subject sequence reads may be used to identify a specimen.

Additional objects, advantages, and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an overview of a collaborative framework suitable for utilizing embodiments of the present invention.

FIG. 2 is a flow chart illustrating a method of obtaining sequence reads from a specimen according to an embodiment of the invention.

FIG. 3 is an illustration of genetic mapping according to an embodiment of the invention illustrated in FIG. 2.

FIG. 4 is a schematic illustration of a computer suitable for use with embodiments of the present invention.

FIG. 5 is a flowchart illustrating a method of identifying sequences within the sample according to an embodiment of the present invention.

FIG. 6 is a flowchart illustrating the Putative Identification of FIG. 5 in accordance with an embodiment of the present invention.

FIG. 7 is a Venn diagram illustrates logic applied to a filtering process according to one embodiment of the present invention.

FIG. 8 is a flowchart illustrating the Mapping Identification of FIG. 5 in accordance with an embodiment of the present invention.

FIG. 9 is a flowchart illustrating the Identification Function of FIG. 5 in accordance with an embodiment of the present invention.

FIG. 10 is a schematic illustration of a fuzzy hash method of filtering and consolidating sequence reads according to embodiment of the present invention.

FIG. 11 is a flowchart illustrating an optional auxiliary process involving how unmapped sequences may be processed according to one embodiment of the present invention.

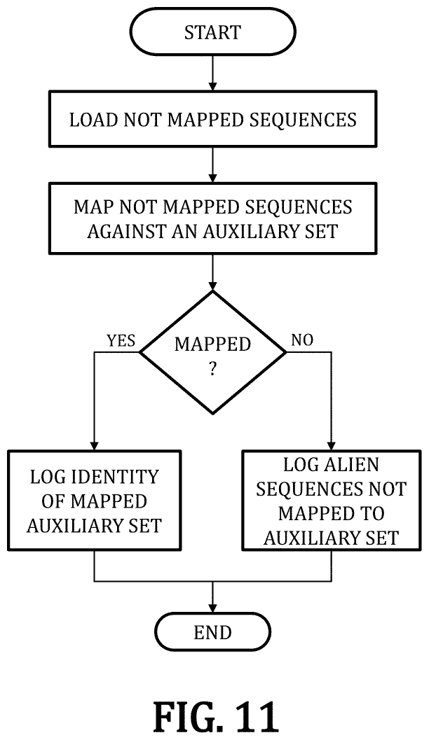

FIG. 12 is an exemplary displayed output according to one embodiment of the present invention.

FIG. 13 is an exemplary displayed output according to one embodiment of the present invention.

FIG. 14 is an exemplary displayed output according to one embodiment of the present invention.

FIG. 15 is a graphical view of taxonomies of sequence reads of a hypothetical read set according to an exemplary embodiment of the present invention.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION OF THE INVENTION

The present invention will now be described more fully hereinafter, including with reference to the accompanying drawings, in which various embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

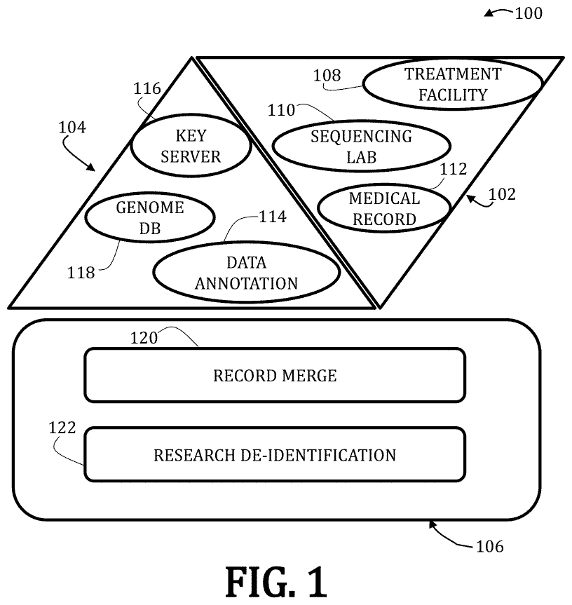

Turning now to the figures, and in particular to FIG. 1, a collaborative framework 100 according to an embodiment of the present invention is shown. The collaborative framework 100 may generally comprise a patient care group 102, a genome annotation group 104, and a genome research group 106. The groups 102, 104, 106 may be particularly arranged so as to minimize risk of personally identifiable information spillage. For example, teams within the patient care group 104 (treatment facility 108, sequencing lab 110, and medical records 112) will require patient name, medical records, medical notes, and so forth. The genome annotation group 104 may further comprise a Data annotation service 114 (configured to be a locus of keys), a key server 116 (configured to key IDs, participant IDs, and encrypt/decrypt keys), and a genome database 118 (configured to encrypt DNA results and associate the encrypted DNA results). The genome research group 106 may include a records merge service 120, which may include information such as patient name, medical record, individual genome, and any identification associated with such patient if included within a particular research project. The genome research group 106 may be further include a research de-identify service 122 for purposes of generating blind studies involving such patient information.

Such proposed separation of roles increases information isolation such that persons within each section of the collaborative framework 100 may only obtain information based on a need to know basis.

For purposes of describing the various embodiments of the present invention, the methods as described herein may be primarily limited to the sequencing laboratory team 110 of the patient care group 102.

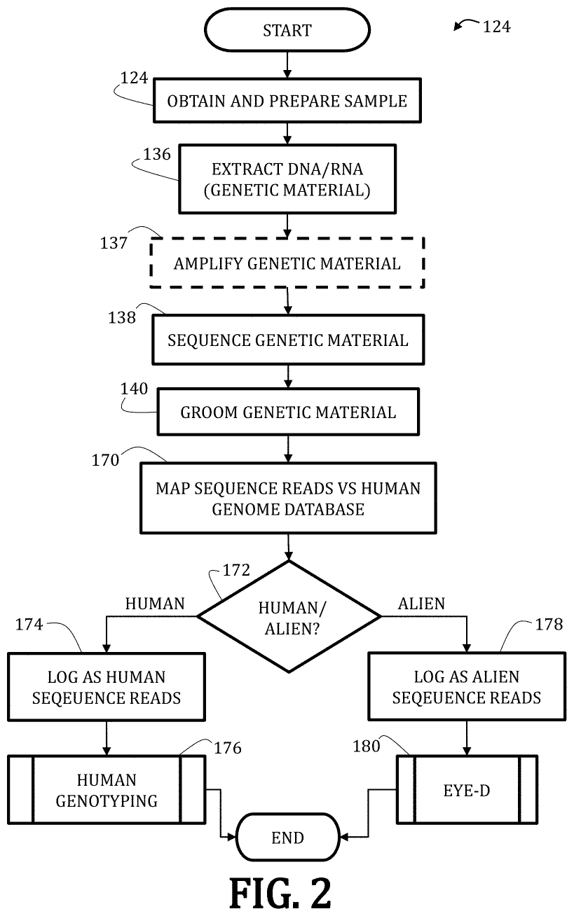

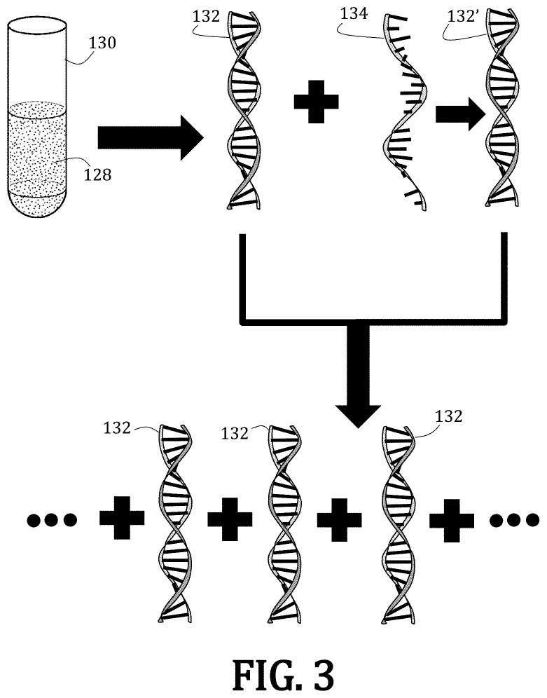

Referring now to FIGS. 2 and 3, a method 124 for obtaining pathogenic sequences according to an embodiment of the present invention is shown. At start, a sample is obtained and prepared (Block 126). The sample may include material obtained from a single organism, a mixture of organisms, the environmental, a food source, an air source, a water source, and combinations thereof. Generally, the sample may be anything that contains intact DNA/RNA, such as dry, fixed, preserved, and fresh specimens. For purposes of illustration, the sample described herein is a biological fluid specimen 128, which may include, but is not limited to, blood or saliva. The specimen 128 may be placed in a suitable container 130 for purposes of analysis as described herein and in a manner that is known to those of ordinary skill in the art of genetics. More particularly, DNA 132, RNA 134, or both may be extracted (Block 136) from the specimen 128. If desired, the strands of RNA 134 may be, optionally, reverse transcribed to strands of DNA 132′. Methods of extraction are known to those of ordinary skill in the art and may include, for example, lysing cells within the specimen 128 (such as by addition of a detergent), degrading (such as with a protease) and precipitating (such as with a salt) DNA 132 and RNA 134, and washing the precipitant. Reverse transcription of RNA 134 to DNA 132′ may include mixing the extracted RNA 132 with primer and reverse transcriptase and incubating, according to any suitable or preferred protocol. In similar manner, although not specifically illustrated herein, proteins and amino acid sequences may be reverse translated to RNA or DNA.

It would be readily appreciated by those or ordinary skill in the art having the benefit of the disclosure made herein that the extracted DNA, RNA, or both (collectively, and hereafter referred to “genetic material”) may originate from various organisms, such as viruses (human pathogens, zoonotic viral pathogens, antiviral resistant gene mutations), bacteria (human pathogens, zoonotic bacterial pathogens, plant diseases, antibiotic resistant strains, virulence factors, toxins), eukaryotes (human parasite and fungal identification, zoonotic parasite and fungal identification, plant parasites, insect subpopulation, tissue-to-species origin, genetically modified organisms, gene doping), or other sources and organisms (barcoding organisms, horizontal gene transfer, genome reorganizations, genome evolution, species and strain evolution, geographic source prediction, human tampering signatures, forbidding gene fusions).

With extraction complete (Block 136), the genetic material may, optionally, be amplified (Block 137) by an appropriate method, such as polymerase chain reaction (“PCR”), sequence amplicons, or fingerprinting products. One suitable PCR protocol, for purposes of illustration, includes initialization, denaturation, annealing, and elongation. More particularly, initialization may include heat activation of the DNA polymerase to denature the DNA. The temperature is lowered to allow annealing of primers, during which primers hybridize to the complementary parts of DNA. Often the temperature is again raised so as to active DNA polymerase is activated to synthesize a new DNA strand, starting at the primer. As a result, a single piece of DNA can be copied thousands to millions of times.

Continuing with reference to FIGS. 2 and 3, the extracted genetic material may be sequenced (Block 138), such as by automated chain-termination DNA sequencing.

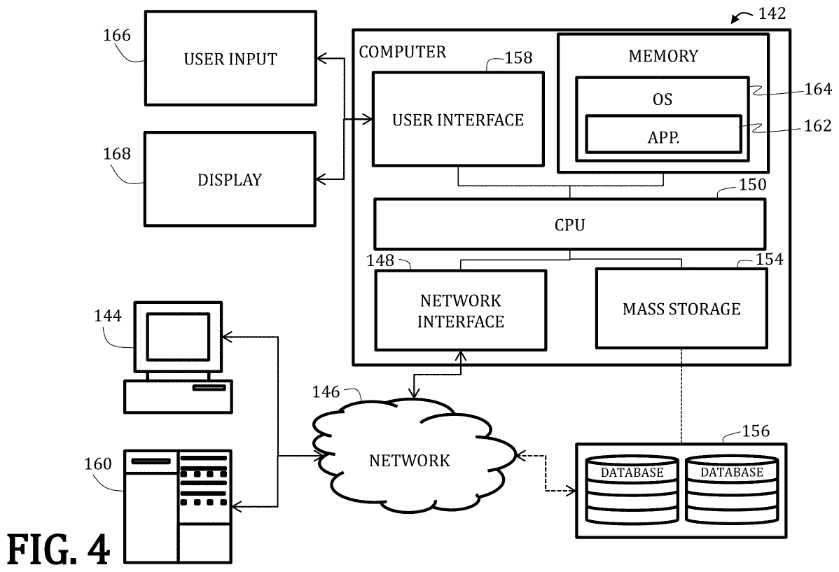

With extraction (Block 136), amplification (Optional Block 137), and sequencing (Block 138) complete, resulting sequences may be prepared for analysis. Analysis may include, according to some embodiments of the present invention, grooming the sequences (Block 140), such as by cleaning, sorting, and so forth, which may be accomplished using a computer 142 (FIG. 4).

As such, and with reference now to FIG. 4, details of the computer 142 for grooming and analyzing the genetic material are described. The computer 142 that is shown in FIG. 4 may be considered to represent any type of computer, computer system, computing system, server, disk array, or programmable device such as multi-user computers, single-user computers, handheld devices, networked devices, embedded devices, etc. The computer 142 may be implemented with one or more networked computers 144 using one or more networks 146, e.g., in a cluster or other distributed computing system through a network interface 148. The computer 142 will be referred to as “computer” for brevity's sake, although it should be appreciated that the term “computing system” may also include other suitable programmable electronic devices consistent with embodiments of the invention.

The computer 142 typically includes at least one processing unit (illustrated as “CPU”) coupled to a memory 152 along with several different types of peripheral devices, e.g., a mass storage device with one or more databases 156, a user interface 158, and the Network Interface 148. The memory 152 may include dynamic random access memory (“DRAM”), static random access memory (“SRAM”), non-volatile random access memory (“NVRAM”), persistent memory, flash memory, at least one hard disk drive, and/or another digital storage medium. The mass storage device 154 is typically at least one hard disk drive and may be located externally to the computer 142, such as in a separate enclosure or in one or more networked computers 144, one or more networked storage devices (including, for example, a tape or optical drive), and/or one or more other networked devices (including, for example, a server 160).

The CPU 150 may be, in various embodiments, a single-thread, multi-threaded, multi-core, and/or multi-element processing unit (not shown) as is well known in the art. In alternative embodiments, the computer 142 may include a plurality of processing units that may include single-thread processing units, multi-threaded processing units, multi-core processing units, multi-element processing units, and/or combinations thereof as is well known in the art. Similarly, the memory 152 may include one or more levels of data, instruction, and/or combination caches, with caches serving the individual processing unit or multiple processing units (not shown) as is well known in the art.

The memory 152 of the computer 142 may include one or more applications 162 (illustrated as “APP.”), or other software program, which are configured to execute in combination with the Operating System 164 (illustrated as “OS”) and automatically perform tasks necessary for processing, analyzing, and grooming sequences with or without accessing further information or data from the database(s) 156 of the mass storage device 154.

A user may interact with the computer 142 via an input device 166 (such as a keyboard or mouse) and a display 168 (such as a digital display) by way of the user interface 158.

Those skilled in the art will recognize that the environment illustrated in FIG. 4 is not intended to limit the present invention. Indeed, those skilled in the art will recognize that other alternative hardware and/or software environments may be used without departing from the scope of the invention.

In any event, referring again to FIG. 2 with the computer 142 of FIG. 4, the sequences may be groomed (Block 140), which may include error corrections, removing background sequence noise, and deleting certain sequences (for example, those that may be related to disease, genetic mutations, privacy information, or controls for which misleading or undesirable results reporting may occur). Some embodiments may preferentially remove genetic material having less than 75 base pairs, low quality bases, low complexity sequences, or combinations thereof. Remaining or resulting groomed genetic materials are, hereinafter, referred to as “sequence reads.”

Thereafter, sequence reads may be categorized as those of human original and those of foreign origin (illustrated as “alien”). Categorization may be accomplished according to one embodiment of the present invention by mapping the sequence reads against one of any number of human genome databases (Block 170), for example, HG 19 or HG 38 (University of California Santa Cruz, Genome Brower, available at http://genome.ucsc.edu). Mapping may be accomplished using one of the various, available resources, such as NextGenMap (GriHub, Inc., San Francisco, Calif.), GEM (Open Source program available at https://github.com/coreyflynn/geneexpressmap), and VelociMapper (TimeLogic, Active Motif Co., Carlsbad, Calif.), to name a few.

Sequence reads associated with the human genome (“Human” branch of Decision Block 172) may be logged as a human sequence read (Block 174) and may be processed according to a human genotyping processes (Block 176). Human genotyping processes (Block 176) may include identification of mutations associated with disease, allelic forming distribution tables, detecting arbitrary genotypes, and research allele discrimination, to name a few. Alien sequences (“Alien” branch of Decision Block 172) may be logged as an alien sequence read (Block 178) may be processed by methods according to embodiments of the present invention, collectively referred to as “Eye-D” (Block 180).

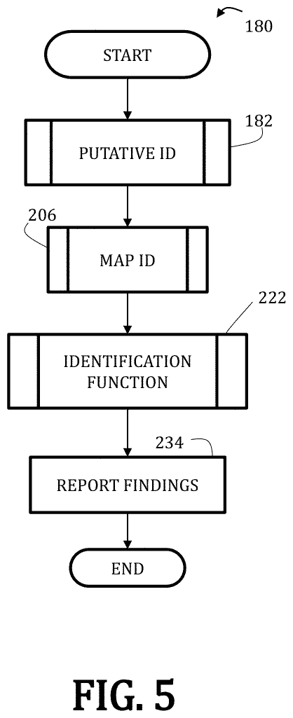

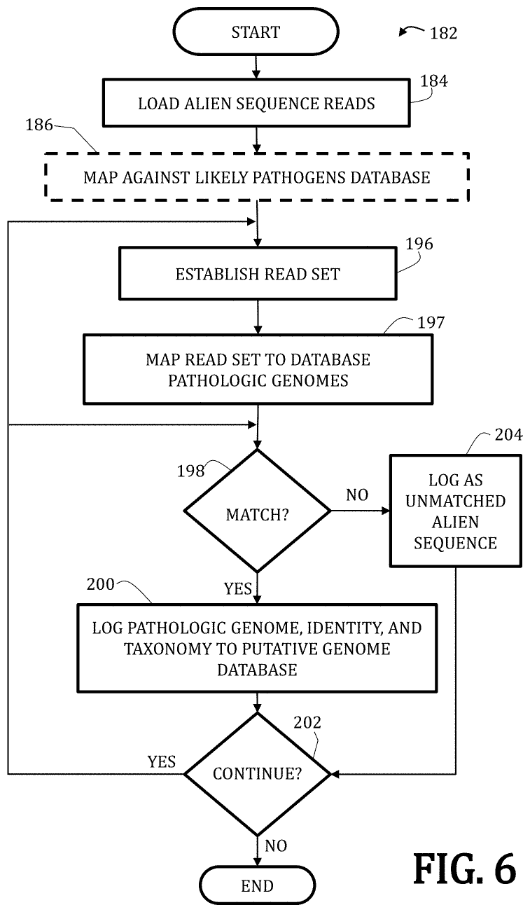

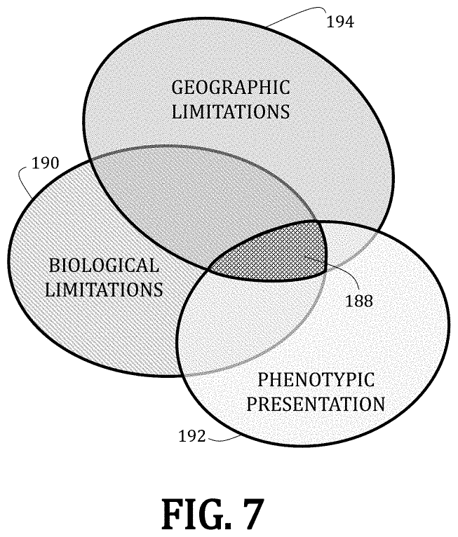

The Eye-D method, illustrated with a flowchart 180 in FIG. 5, begins with a putative ID process 182, which is, itself, illustrated according to one embodiment of the present invention in FIG. 6. In that regard, alien sequences may be loaded into memory 152 (FIG. 4) (Block 184). Optionally, the sequence reads may be compared to a database comprising likely pathogen sequences (Optional Block 186). Such likely pathogen database may be tailored so as to be a best guesses, by eliminating those virulent strains that are unlikely (whether due to geographic limitations or phenotypic presentations), or a combination thereof. For example, in the Venn Diagram of FIG. 7, an intersection 188 of various criteria may yield a subset of sequences that is more likely to map to at least one of the alien sequence reads. Such criteria may be based, for example, biological limitations 190 (based on sex, race, strain, and so forth), phenotypic presentation 192 (observable presentations), and geographic limitations 194 (areas of exposure or area sample collection). According to other embodiments, the likely pathogen database may comprise a sequences relating to pathogen for which mere detection is desired. For example, if knowledge of the presence of F. tularensis is desired, then the genome of F. tularensis may be included. In this way, computing resources may be minimized, which facilitates in-the-filed applications. Alternatively, or additionally, the likely pathogen database may comprise a specifically curated target database having genomes of particular national security interest, such as known biological warfare agents.

Referring again to FIG. 6, the loaded alien sequence reads may be sampled to establish a read set (Block 196). The sampling may, according to some embodiment so the present invention, be random. Moreover, the number of alien sequence reads comprising the read set may vary and may depend largely on a number of alien sequence reads logged (Block 178, FIG. 2). According to some embodiments, a number of sequence reads comprising the read set may be 1000; however the number of sequence reads may alternative range from, for example and without limitation. Another manner by which to limit a number of reads for the read set may be by computation load. Thus, some embodiments may limit the read set to 1 Mb. Alternatively, sampling of the alien sequence read may continue, such as iteratively, until no new sequence read is sampled within a defined number of sampling iterations. Such sampling of the loaded alien sequence reads further minimizes computational load by significantly reducing a number of sequence mappings as described in detail below.

With the read set established, the read set may be mapped (Decision Block 198) to a database comprising pathologic genomes. Sequence mapping may include any one from a variety of methods used by those of ordinary skill in the art (for example, CLUSTALX, which is an open source freeware). The database may include publicly known pathologic genomes, pathologic genomes of national security interest, pathologic genomes of proprietary interest, other suitable pathologic genomes, and combinations thereof. Suitable databases may include, for example, broad resources (such as a derivative of GENBANK using the National Center for Biotechnology Information (“NCBI”) Basic Alignment Search Tool (“BLAST”) or the Bowtie 2 (Johns Hopkins University, Baltimore, Md.)) to narrowly defined investigations tailored to specific pathogen identification (for example, F. tularensis or registered, select agents). Moreover, one or more of these pathologic genomes may be tailored in a manner as described above with reference to FIG. 7. That is, to reduce computational load, the one or more pathologic genomes comprising the database may be filtered or refined based on criteria (for example, and without limitation, the criteria 188, 190, 192 described above). Additionally or alternatively, if any sequence reads mapped against likely pathogens (Block 184), then the genomes of the respective pathogens may be removed from the database. According to other embodiments, sequences associated with the taxa of the specimen host may be removed; however, such sequences may be maintained for purposes of investigating order level lateral gene transfers, duplications, translocations, or combinations thereof, for example.

When a sequence read from the read set maps to a portion of one or more genomes within the database with a certainty above a selected threshold (for example, at least 98% confidence, a MapQ10 corresponding to greater than 90% identity, or MAPQO indicating two or more identical matches) (“YES” branch of Decision Block 198), then the one or more genomes, the organism identity of the respective one or more genomes, and the taxonomic tree of these organism identities may be logged to a putative genome database (Block 200). Optionally, the genomes, identity, and taxonomic tree of genomes or organisms considered to be equivalent to a logged genome may also be logged. According to yet other embodiments of the present invention, particularly those focused on further reducing computational load, the entire taxonomies may be downloaded at a later time such that the putative genome database requires smaller amounts of computer memory. The process may continue (“YES” branch of Decision Block 202) if sequence reads remain in the read set by returning for further mapping (Decision Block 198). Alternatively, if no more sequences reads remain in the read set, but additional investigation is desired, the process may return to the selection sequence reads (Block 196). Otherwise, the process may end (“NO” branch of Decision Block 202). Alternatively still, continuation may be necessary or desired when new matches or correlations between the alien sequence reads sequences, not previously included in the read set, maps to at least a portion of a genome of the database.

For those sequence reads that do not map to any portion of the one or more genomes within the database (“NO” branch of Decision Block 198), then the sequence read may be logged as an unmatched alien sequence and removed from the read set (Block 204). The process may continue (Decision Block 202) as described above.

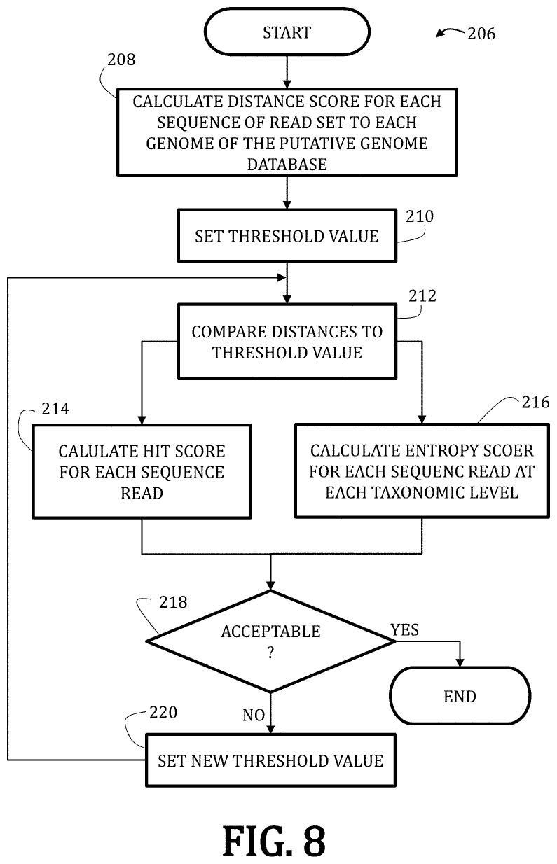

Returning again to FIG. 5, and with the putative ID process complete (Block 182), a map ID process may begin (Block 206), which is illustrated with greater detail in FIG. 8. At start, although not specifically shown, the putative genome database and the read set are loaded into memory 152 (FIG. 4). Each sequence of the read set may be compared to each genome of the putative genome database such that a distance score may be assigned thereto (Block 208). The distance score may be a quantitative value that represents a level of similarity between each sequence of the read set and each genome of the putative genome database. According to one particular embodiment of the present invention, the distance score may be a percent of homology. According to the illustrative embodiment, the distance score is determined by comparing a number of hydrogen bonds comprising the sequences. More specifically, and as would be understood by those having ordinary skill in the art, hydrogen bonds bind the two strands of DNA together according to Watson-Crick base pairs: adenine to thymine having two hydrogen bonds while guanine and cytosine have three hydrogen bonds therebetween. As a result, each unique sequence of Watson-Crick base pairs will have an integer number of base pairs. Thus, a distance score is the comparison of the numbers of hydrogen bonds of each sequence of the read set and a mapped portion of each genome of the putative genome database.

According to other embodiments of the present invention, the distance score may be calculated in another way. For example, BLAST methodology includes a BLAST score; other methodologies include BOWTIE. In effect, any methodology may be used so long as the score is proportional to a length of the read and an accuracy of the match between the sequence read and the genome.

With distance scores calculated, a threshold of permitted difference between the sequences of the read set and the genomes of the putative genome database is set (Block 210). While the threshold may vary, suitable thresholds may be, for example 80%, 85%, 90%, 95%, or 98%. Comparisons having distance scores less than the threshold are thus deemed to be insufficiently mapped to warrant further analysis or to identify that putative organism as being present in the sample.

According to some embodiments of the present invention, the threshold may be customized to the type of genome considered. For example, it would be appreciated by the skilled artisan that a variation in bacteria is less than a variation in viruses; therefore, the threshold level for mapping to bacterial-based genomes may be less than the threshold level for mapping to viral-based genomes.

In Block 212, each distance score is then compared to the threshold for calculating a hit score (Block 214) and an entropy score (Block 216).

The hit score (Block 214) may be a summation of the binary response to the comparison between the distance score and the threshold. In other words, for each sequence of the read set having a distance score greater than the threshold value, a “hit” may be recorded (integer value of “1”). For each sequence of the read set having a distance score less than the threshold value, no hit is recorded (integer value of “0”). Thus, the hit score may be considered a number of threshold hits a sequence of the read set has to the genomes of the putative genome database.

The entropy score (Block 216) may be a measure of how sequences of the read set have a biologically relevant hit score. Such that perfectly unique hit of one sequence of the read set to exactly one genome of the putative genome database will have an assigned entropy score of 1. Inexact mapping, or multiple mappings will thus, by definition, have an entropy score that is greater than 1. In that regard, the entropy score may be calculated by reviewing the hit score at each taxon level. If a sequence of the read set has a distance score greater than the threshold value and having an appropriate taxon level (whether the genome of a species, genus, family, order, and so forth), then an entropy hit may be recorded (integer value of “1”). If the sequence of the read set has a distance score less than the threshold value OR the taxon level differs, then not entropy hit is recorded (integer value of “0”).

The least common root taxonomic group that contains all of the hits that yield an entropy score greater than 1 will be the greatest common taxonomic assignment possible for a given sequence.

With distance scores and entropy scores determined for all sequences of the read set, a determination as to whether sufficient information is resulted is made (Decision Block 218). If such data is sufficient (“YES” branch of Decision Block 218), then the process may end and return to FIG. 5; however, if such data is insufficient (“NO” branch of Decision Block 218), then a threshold value made be set (Block 220) and the process returns to compare distances to the newly set threshold value (Block 212) such that new hit scores and entropy scores may be calculated. Sufficiency of the data may be determined by evaluating the hit scores and the entropy scores. For instance, if few-to-no hits are made (evidenced by low hit scores or no, non-zero hit scores), then the threshold value set in Block 210 may be too great and a lower threshold value should be set in Block 220. Another example may be if the entropy scores remain high over several taxon levels such that little distinction between members of the same order, the same family, or the same genus can be made in view of the threshold value. Generally, with respect to the entropy score, determining to alter the threshold value may include considering a difference in the distance score between a best matching member of a taxon group and a worst matching member of a taxon group. If the difference in distance score is large, then threshold value may need to be increased to further filter outliers. If the difference in distance score is small, then the threshold value may need to be decreased to capture greater diversity.

If any sequence of the read set maps to more than one genome of the putative genome database at the species taxon level (or more particularly, such as a subspecies or strain), then it is likely that such sequence is not diagnostic of a strain or species; however, the hit score, entropy score, and sequence mapping may still be logged.

Although not specifically illustrated in FIG. 8, for any sequence of the read set that does not map to at least one sequence of the putative genome database, the sequence read, its hit scores may be logged as “not mapped” for further and later analysis.

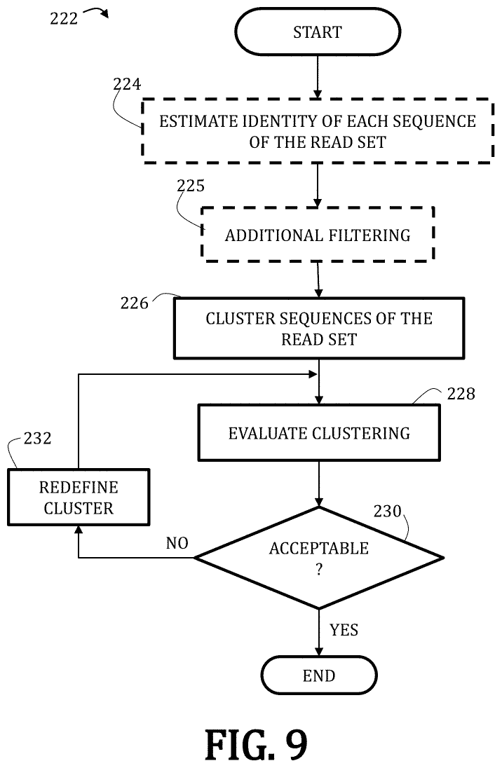

Returning once again to FIG. 5 and with the map ID process complete (Block 206), the process may continue to an identification function (Block 222), which is illustrated in FIG. 9. Sequence reads having diagnostic value may be identified as those having a low, final entropy score (preferably, an entropy score of 1). However, the final entropy score is often an “average” entropy score that describes genetic variation of the particular organism. For instance, it would be readily appreciated that some regions an organism's genome may be more naturally prone to variation than others.

In that regard, at start, and if desired, an estimation of the identity for each sequence of the read set may be made (Block 224). The estimation may include an evaluation of the hit score and the entropy score of each read—if sufficient data is present (such as an entropy value of 1 for a species), then the identity of the organism from which the sequence was obtained may be known at the level of certainty set by the threshold (Block 210 or Block 220 of FIG. 8). In some embodiments, the absence of hit score, entropy score, or both may be indicative of the lack of sequences from a designated organism, which may satisfactory. For example, if no hit score, no entropy score, or both are calculated against the SARS coronavirus genome, then the estimation may be that SARS coronavirus was not present in the specimen.

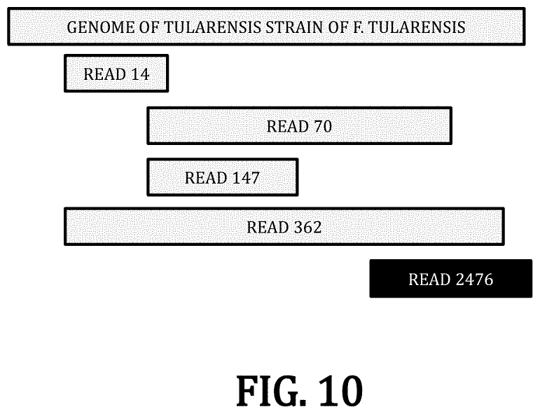

In the interest for further reducing computational load, the number of sequences comprising the read set may be further reduced by filtering (Optional Block 225). According to one embodiment illustrated in FIG. 10, a fuzzy hash method may be used. In FIG. 10, the genome of the tularensis strain of F. tularensis is shown in toto and in block format. Sequence reads 14, 70, 147, 362, and 2476 of a read set (not shown in FIG. 10) map to at least a portion of the F. tularensis genome. Based on hit scores and entropy scores, reads 14, 70, 147, and 362 have been tentatively designated as mapping to F. tularensis, tularensis; however, read 2476 was tentatively designated as mapping to a species of bacteria that is not directly related to F. tularensis, tularensis. As a result, reads 14, 70, and 147 may be filtered from the read set or, considered another way, collectively represented by read 362. Read 2476 remains separate for further analysis. In this way, the number of sequence reads comprising the read set may be further reduced with a degree of certainty. Such reduction not only further reduces computational load but may significantly reduce a number of results to be reviewed in a final reporting.

In a similar fashion, it would be readily appreciated by those having ordinary skill in the art having the benefit of the disclosure made herein that a genome need only be identified once with a given level of certainty for a conclusion that the organism represented by the genome was present in the sample.

After optional estimation or filtering, the process may continue to clustering the sequences in a manner that maximizes certainty to a read's identity (Block 226). In effect, sequence reads of the filtered read set may be grouped together such that a combined hit score, a combined entropy score, and a diversity in distance score (hereafter referred to as “ADistance”) may be calculated. Thus, each sequence read may only exist in one cluster at a time so that its distance score, entropy score, and so forth contribute to a singular score for the respective cluster.

In effect, the sequences of the read set may be clustered in a combinatorial optimization manner. Sequences of the read set may be clustered or unclustered in any manner so as to minimize ADistance of the clusters and maximize the vote. Thus, if the addition of a sequence to previously formed cluster reduces the cluster hit score, then it is likely that the sequence does not belong within the cluster. Increases in a cluster hit score preferred over increases in ADistance.

Clustering according to Block 226 may begin with the clustering of a highest taxon tiers (such as subspecies or species) and may move upwardly through the taxonomy of each sequence. For example, if a sequence originated from a widely dispersed species (a plant gene, for example, should not be found in a bacteria genome), then the entropy score of a cluster having both the plant and bacteria sequence will be more strongly skewed upwardly less because such horizontal gene transfer would not be likely and would typically require more mutations. Conversely, a bio-engineered bacteria may exhibit exaggerated ADistance when compared to a phylogenetically close relatives. Such alterations may be of significant interest and may be logged.

With clustering, the cluster hit score may be used to weigh the hits toward members of a given, putative unknown that is more similar to a sequence so as to minimize ΔDistance with respect to the collection of hits as correlated to the magnitude of the hit score. For example, such could be in a manner similar to K means clustering the multiplicative inverse of the hit score or using a Modulo operation. As clustering moves from highest to lowest tiers (for example, from species to kingdom or root), the hit score may be penalized as:

E=10nT Equation 1

wherein E is the hit score, n is the number of mapped hits, and T is the least common taxon tier. Accordingly, a hypothetical, novel species may have a large distance from the greatest common taxonomic group if there are more hits (high entropy score) or the hit scores are, on average, lower.

As clusters are formed and scores recalculated, there is a determination whether a redefined (or new) cluster improves scores by maximizing hit score and minimizing ΔDistance (Decision Block 230). If such cluster does not so improve the hit score or another clustering strategy is desired (“NO” branch of Decision Block 230), then there may be another redefining of the cluster (Block 232), and the process returns to evaluate the newly redefined cluster (Block 228). If clustering is complete (“YES” branch of Decision Block 230), then the process may end and return to FIG. 5.

The desired end point of the Eye-D method 180 of FIG. 5 is to find the names of organisms found within the specimen. The clustering, maximizing of hit score, and minimizing of ΔDistance according to the embodiments herein is to identify the least number of results that contain all of the high probability taxonomic elements. Thus, with identities, or lack thereof, determined, findings of the Eye-D method 180 may be reported (Block 234). The report may be formal or informal and may include a range of information, such as sequence alignments, conventional phenotypic or clinical presentations, degree of certainty, number of base pairs mapped, taxonomy information, phylogenetic trees, and so forth. Exemplary reports are illustrated in Example 1, below; however, such reports are illustrative only and should not be considered to be limiting.

While not specifically illustrated herein, the non-mapping sequences noted above, may be subject to further analysis. In that regard, the non-mapping sequences may be mapped against an auxiliary set of sequences. Exemplary auxiliary sets of sequences may include protein sequences, motif sequences, toxin-virulent sequences, controlled databased of warfare sequences, or a combination thereof. In each of these embodiments, mapping of the non-mapping sequence read may be attempted against genomes or sequences of the auxiliary set of sequences. For any sequence mapping with a certainty above the selected threshold, the identity of the respective pathogen may be reported as being present within the specimen. Otherwise, the sequences not mapping to the loaded auxiliary set of sequences may be examined against another auxiliary set. While the use of such auxiliary sets of sequences may operate in a sequential manner, it would be understood by those having ordinary skill in the art and the benefit of the disclosure provided herein that the order of mapping and number of auxiliary sets need not be limiting.

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

### Example 1

Using a methodology according to an embodiment of the present invention described herein, a number of PCR and full genome amplification products were identified. The tests amplified large sections of related viral pathogens through the use of degenerate PCR of specially selected locations in the viral genome using first and second primers. After PCR amplification, resulting products were subjected to direct sequencing with a third primer (similar to one of the prior two) to provide sequences ranging from 25 base pairs to 600 base pairs, depending on the downstream instrument used. The locations chosen for the specific amplicons met several very specific guidelines and were selected via computer assistance. The goal was to select regions of strong biological conservation (sequence similarity) that flanked regions of strong divergence. This maximizes the diversity observed in the sequence tag.

PCR and sequencing were accomplished per the respective vendors' product protocols. The yielded bases were examined and all detections were made autonomously. In all cases, the sequence was automatically submitted for analysis via direct laboratory networking.

Variability of a divergent region acted as a “DNA barcode,” requiring no further manipulation to determine a nature of the organism. The sequence (in few bases of conserved zone) readily showed the organism major group (usually genera). The exact sequence in divergent zones provided the strain identification. If a related sequence region was obtained and paired with an unknown divergent zone, then a new strain was identified. Known strains generally matched the selected database. Average limits of detection were below 100 genome equivalents for most virus strains used. Sequencing does not appear to alter the limits of detection.

To test the identifying of novel targets according to embodiment of the present invention, a deletion test was performed. Specific strains were removed from the database. Sequencing results were then used to infer the proper taxonomic assignment. Autonomous tests showed greater than 98% accuracy, which was in line with the predicted Q20 (99%) predicted accuracy of name. The procedure was seen to readily detect both known (in database) and unknown organisms (synthetic DNA or left out of database) in each of these major viral classes. The tests correctly identified serotype co-detections in both spiked and unknown clinical samples. The method can detect simulated emergent infections (synthetic DNA simulants) and even natural drift in ATCC stock strains when compared to GENBANK.

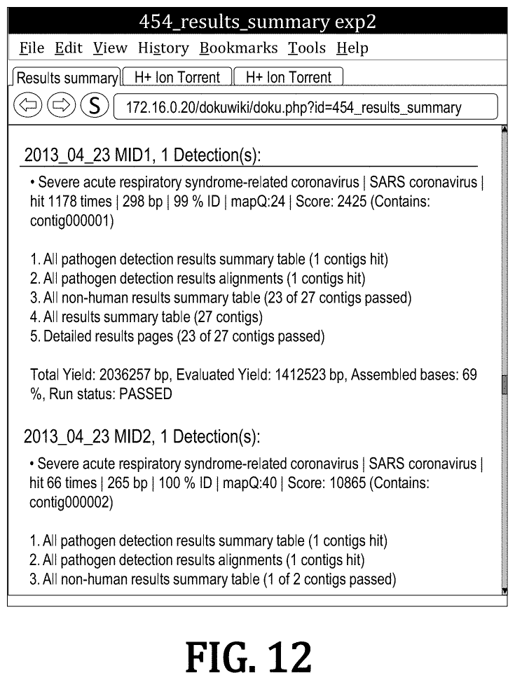

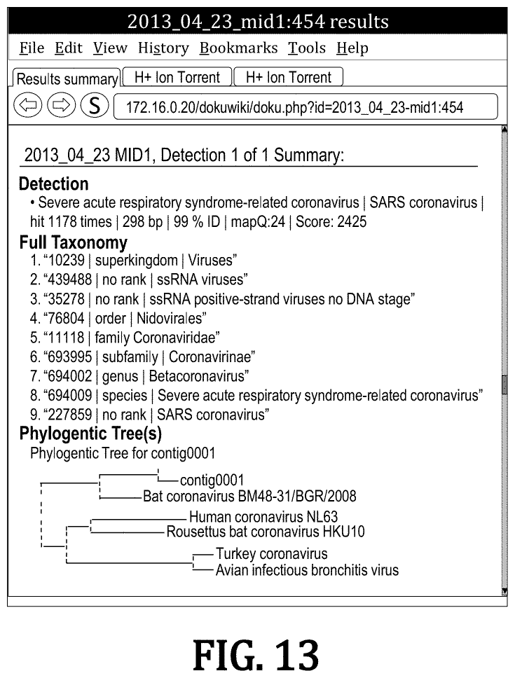

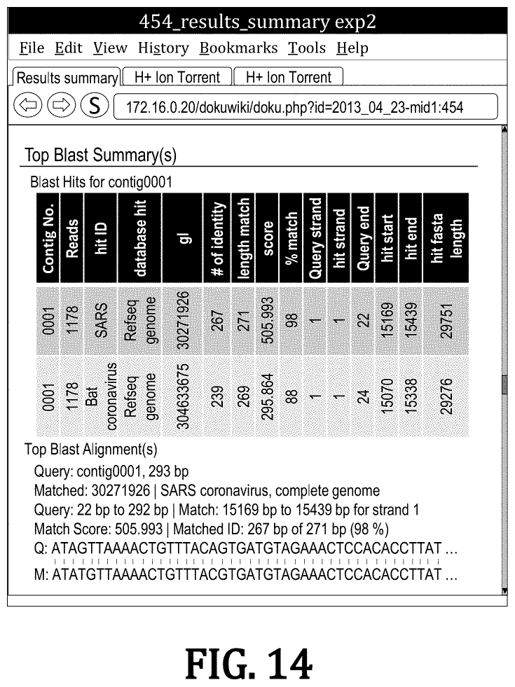

FIG. 12 is an exemplary screen shot in which single line pathogen detections within the specimen are presented to a user. FIG. 13 is an exemplary screen shot in which automated ID and taxonomy tree placement based on resulting sequences are presented to a user. FIG. 14 is an exemplary screen shot in which alignment and quality of match are presented to a user. Additional reporting may include, but is not limited to, figures of genome coverage or gene variation reports.

### Example 2

Assuming a sample was prepared, sequenced, and groomed according to the illustrative embodiment of FIG. 2, a sampling of the sequence reads resulted in a read set comprising Sequence Read Nos. 1, 10, 14, 21, 23, 26, 32, 35, 39, 40, 41, 43, 54, 59, 63, 68, 72, 85, 88, 89, 96, and 98 of the original 120 sequences.

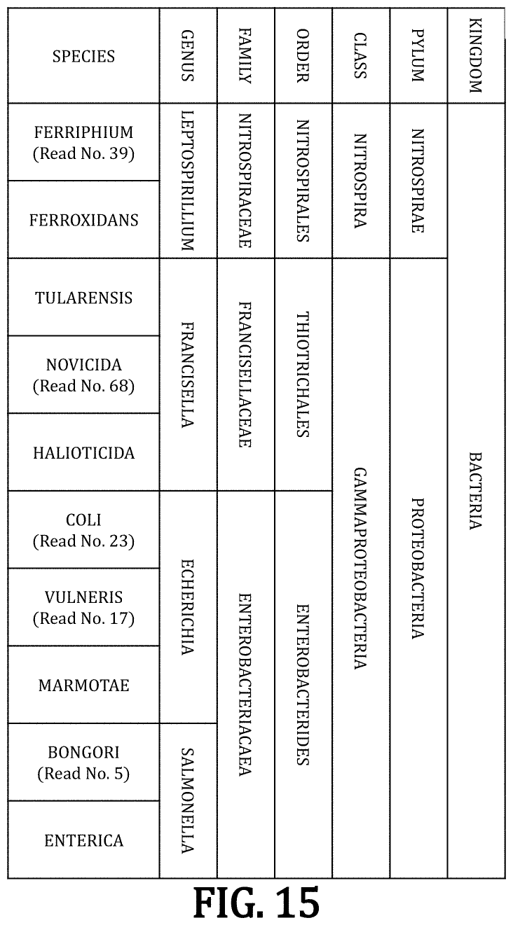

Mapping of these sequences of the read set against an omnibus genome database yielded a putative genome database comprising Putative Genome Nos. 1-19. The organism identification and taxon level for each genome of the putative genome database is provided in Table 1, below. Full taxonomy information is provided in FIG. 15.

Assuming each sequence of the read set has 200 hydrogen bonds, hypothetic distance scores are provided in Table 2.

Distance scores were calculated for threshold values of 80%, 85%, 90%, 95%, and 98% and are shown in Table 3, below.

Exemplary entropy scores for Seq. Read Nos. 1 and 68 are shown in Tables 4 and 5, respectively, below.

TABLE 1

| Putative Genome No. | Identification | Taxon level |
|---|---|---|
| 1 | L. ferriphium | Species |
| 2 | Salmonella | Genome |
| 3 | F. tularensis | Species |
| 4 | F. novicida | Species |
| 5 | S. bongori | Species |
| 6 | Enterobacteriaceae | Family |
| 7 | Enterobacterides | Order |
| 8 | E. marmotae | Species |
| 9 | Echerichia | Genus |
| 10 | S. enterica | Species |
| 11 | Leptospirillium | Genus |
| 12 | L. ferroxidaris | Species |
| 13 | Francisella | Genus |
| 14 | Thiotrichales | Order |
| 15 | F. halioticida | Species |
| 16 | E. coli | Species |
| 17 | E. vulneris | Species |
| 18 | Francisellaceae | Family |
| 19 | Gammaproteobacteria | Class |

TABLE 2
DISTANCE SCORES

| | | PUTATIVE GENOME NO. | | | | | | | | | |
|---|---|---|---|---|---|---|---|---|---|---|---|
| | | 1 | 2 | 3 | 4 | 5 | 6 | 7 | 8 | 9 | 10 |
| SEQUENCE | 1 | 197 | 5 | 36 | 154 | 42 | 84 | 85 | 129 | 86 | 28 |
| READ | 10 | 105 | 193 | 190 | 193 | 196 | 193 | 191 | 190 | 192 | 190 |
| NO. | 14 | 31 | 191 | 192 | 195 | 190 | 191 | 194 | 192 | 194 | 192 |
| | 21 | 8 | 192 | 190 | 195 | 195 | 190 | 191 | 195 | 191 | 195 |
| | 23 | 43 | 193 | 191 | 190 | 192 | 190 | 192 | 193 | 190 | 195 |
| | 26 | 2 | 192 | 194 | 190 | 197 | 190 | 193 | 190 | 192 | 192 |
| | 32 | 39 | 192 | 195 | 194 | 193 | 193 | 194 | 194 | 193 | 193 |
| | 35 | 96 | 192 | 192 | 193 | 194 | 195 | 192 | 190 | 193 | 194 |
| | 39 | 199 | 2 | 46 | 124 | 96 | 93 | 86 | 129 | 107 | 98 |
| | 40 | 88 | 194 | 195 | 190 | 198 | 195 | 190 | 190 | 194 | 194 |
| | 41 | 136 | 192 | 191 | 190 | 191 | 191 | 195 | 192 | 190 | 191 |
| | 43 | 92 | 193 | 192 | 197 | 193 | 191 | 193 | 193 | 192 | 191 |
| | 54 | 12 | 190 | 195 | 193 | 193 | 194 | 194 | 192 | 194 | 190 |
| | 59 | 74 | 192 | 190 | 194 | 192 | 195 | 192 | 191 | 191 | 191 |
| | 63 | 64 | 195 | 194 | 194 | 196 | 191 | 195 | 195 | 192 | 10 |
| | 68 | 124 | 195 | 195 | 198 | 193 | 194 | 193 | 194 | 195 | 191 |
| | 72 | 34 | 193 | 190 | 192 | 193 | 190 | 195 | 192 | 195 | 193 |
| | 85 | 195 | 35 | 128 | 160 | 24 | 136 | 38 | 26 | 98 | 77 |
| | 88 | 119 | 190 | 194 | 191 | 190 | 194 | 190 | 193 | 190 | 193 |
| | 89 | 16 | 192 | 191 | 193 | 199 | 190 | 191 | 194 | 195 | 195 |
| | 96 | 27 | 194 | 190 | 196 | 193 | 195 | 195 | 192 | 194 | 191 |
| | 98 | 95 | 193 | 194 | 190 | 195 | 195 | 190 | 193 | 190 | 194 |

| | | PUTATIVE GENOME NO. | | | | | | | | |
|---|---|---|---|---|---|---|---|---|---|---|
| | | 11 | 12 | 13 | 14 | 15 | 16 | 17 | 18 | 19 |
| SEQUENCE | 1 | 199 | 191 | 118 | 1 | 57 | 33 | 136 | 135 | 125 |
| READ | 10 | 138 | 79 | 195 | 194 | 195 | 192 | 192 | 190 | 193 |
| NO. | 14 | 0 | 59 | 193 | 195 | 192 | 194 | 196 | 190 | 194 |
| | 21 | 24 | 89 | 195 | 194 | 190 | 190 | 193 | 193 | 194 |
| | 23 | 152 | 13 | 193 | 195 | 195 | 199 | 190 | 193 | 194 |
| | 26 | 40 | 2 | 193 | 192 | 192 | 193 | 194 | 195 | 194 |
| | 32 | 132 | 5 | 194 | 192 | 192 | 193 | 198 | 191 | 195 |
| | 35 | 126 | 11 | 194 | 193 | 194 | 196 | 191 | 194 | 192 |
| | 39 | 191 | 193 | 69 | 55 | 110 | 98 | 134 | 119 | 40 |
| | 40 | 140 | 57 | 191 | 193 | 195 | 190 | 191 | 190 | 190 |
| | 41 | 65 | 122 | 195 | 193 | 191 | 192 | 198 | 190 | 191 |
| | 43 | 31 | 96 | 194 | 191 | 191 | 194 | 193 | 192 | 194 |
| | 54 | 38 | 40 | 194 | 195 | 193 | 197 | 195 | 195 | 198 |
| | 59 | 3 | 5 | 195 | 195 | 193 | 197 | 193 | 192 | 193 |
| | 63 | 46 | 2 | 191 | 190 | 192 | 190 | 192 | 195 | 194 |
| | 68 | 65 | 53 | 198 | 200 | 193 | 193 | 193 | 199 | 198 |
| | 72 | 79 | 68 | 190 | 194 | 193 | 195 | 196 | 195 | 192 |
| | 85 | 193 | 193 | 46 | 28 | 45 | 65 | 136 | 25 | 68 |
| | 88 | 82 | 126 | 192 | 195 | 190 | 198 | 192 | 194 | 194 |
| | 89 | 156 | 53 | 190 | 195 | 195 | 191 | 193 | 195 | 194 |
| | 96 | 10 | 152 | 191 | 192 | 190 | 192 | 190 | 190 | 195 |
| | 98 | 10 | 138 | 192 | 190 | 194 | 197 | 192 | 194 | 194 |

TABLE 3
Hit scores

| SEQ. READ NO. | 80% | 85% | 90% | 95% | 98% |
|---|---|---|---|---|---|
| 1 | 3 | 3 | 3 | 3 | 2 |
| 10 | 16 | 16 | 16 | 12 | 1 |
| 14 | 16 | 16 | 16 | 14 | 1 |
| 21 | 16 | 16 | 16 | 12 | 0 |
| 23 | 16 | 16 | 16 | 12 | 1 |
| 26 | 16 | 16 | 16 | 13 | 1 |
| 32 | 16 | 16 | 16 | 16 | 1 |
| 35 | 16 | 16 | 16 | 15 | 1 |
| 39 | 3 | 3 | 3 | 3 | 1 |
| 40 | 16 | 16 | 16 | 10 | 1 |
| 41 | 16 | 16 | 16 | 13 | 1 |
| 43 | 16 | 16 | 16 | 16 | 1 |
| 54 | 16 | 16 | 16 | 14 | 1 |
| 59 | 16 | 16 | 16 | 15 | 1 |
| 63 | 16 | 16 | 16 | 13 | 1 |
| 68 | 16 | 16 | 16 | 16 | 5 |
| 72 | 16 | 16 | 16 | 13 | 1 |
| 85 | 3 | 3 | 3 | 3 | 0 |
| 88 | 16 | 16 | 16 | 11 | 1 |
| 89 | 16 | 16 | 16 | 14 | 1 |
| 96 | 16 | 16 | 16 | 12 | 1 |
| 98 | 16 | 16 | 16 | 12 | 1 |

TABLE 4
Entropy scores for SEQ. READ NO. 1

| | Kingdom | Phylum | Class | Order | Family | Genus | Species |
|---|---|---|---|---|---|---|---|
| @ 80% | 1 | 1 | 1 | 1 | 1 | 1 | 2 |
| @ 85% | 1 | 1 | 1 | 1 | 1 | 1 | 2 |
| @ 90% | 1 | 1 | 1 | 1 | 1 | 1 | 2 |
| @ 95% | 1 | 1 | 1 | 1 | 1 | 1 | 1 |
| @ 98% | 1 | 1 | 1 | 1 | 1 | 1 | 1 |

TABLE 5
Entropy scores for SEQ. READ NO. 68

| | Kingdom | Phylum | Class | Order | Family | Genus | Species |
|---|---|---|---|---|---|---|---|
| @ 80% | 1 | 1 | 1 | 1 | 1 | 1 | 3 |
| @ 85% | 1 | 1 | 1 | 1 | 1 | 1 | 3 |
| @ 90% | 1 | 1 | 1 | 1 | 1 | 1 | 3 |
| @ 95% | 1 | 1 | 1 | 1 | 1 | 1 | 2 |
| @ 98% | 1 | 1 | 1 | 1 | 1 | 1 | 1 |

While not specifically shown, fuzzy hash clustered sequence reads as provided in Table 6. The representative sequence for each of the five estimated identities is noted with an asterisk, *.

TABLE 6

| Sequence Read No. | Estimated identification |
|---|---|
| 1 | L. ferriphium |
| 10 | S. bongori |
| 14 | E. vulneris |
| 21 | F. novicida |
| 23 * | E. coli |
| 26 | S. bongori |
| 32 | E. vulneris |
| 35 | E. coli |
| 39 * | L. ferriphium |
| 40 | S. bongori |
| 41 * | E. vulneris |
| 43 | F. novicida |
| 54 | E. coli |
| 59 | E. coli |
| 63 | S. bongori |
| 68 * | F. novicida |
| 72 | E. vulneris |
| 85 | L. ferriphium |
| 88 | E. coli |
| 89 * | S. bongori |
| 96 | F. novicida |
| 98 | E. coli |

From the above data, it may be concluded that Sequence Read No. 1 originated from a single species with 95% certainty—the species corresponding to Putative Genome No. 1, which is L. ferriphium. Likewise, Sequence Read No. 68 originated from a single species with 95% certainty—the species corresponding to Putative Genome No. 1, which is L. ferriphium.

### Example 3

A plurality of sequence reads were obtained from sequencing the DNA and RNA of a sample. A read set comprising 6648 sequences was obtained from the plurality of sequence reads. Prior to evaluating the read set against an omnibus database comprising a plurality of genomes, a filter was applied to the omnibus database. Criteria for the filter may be found in Table 7. Therein, a filter type is defined with one or more instructions therein. For instance, the #controls filter included two instructions: filter out genomes and sequences associated with (1) Taxon ID #1246486, which is associated with synthetic Enterobacteria phase phiX174.1f and (2) Taxon ID #10842, which is associated with microvirus. The #Insects & mites & ectoparasites filter includes several instructions of one of two type: filter out or include. The #Insects & mites & ectoparasites filters out sequences associated with Taxon ID #6656, which is associated with Arthropoda, generally. However, pathogenic arthropods (such as pediculus, culicidae, and so forth) are retained within the omnibus database.

Table 8 is a truncated set of sequences of the read set. Sequence 7257 hit one genome of the putative genome database six times—thus, 6 hits to Taxon Code 11128 (the putative genome database ID being 15081544), which is the complete genome of the bovine coronavirus. Because only one taxon group was hit by this sequence, the entropy score of Sequence 7257 is 1.

Referring still to Table 8, Sequence 8369, unlike Sequence 7257, mapped to several genomes of the putative genome database. For instance, Sequence 8369 mapped to Taxon code 408 (the complete genome of Methylobacterium extorquens strain PSBB040) and Taxon code 1076. However, Taxon code 1076 identifies both (1) whole genome shotgun sequence of Rhodopseudomonas palustris strain 420L contig 45 and (2) whole genome shotgun sequence of Rhodopseudomonas palustris strain BAL298 c293|2759c662.853943. As result of these two examples, the hit score for Sequence 8369 is increased by 5 for the five hits to Taxon code 408 and is increased by 2 for the two hits to Taxon code 1076. However, the entropy score for Sequence 8369 is increased by only 1 for Taxon code 408 because these hit were all at the same taxon level while the entropy score is increased by 2 for Taxon code 1076 because two different strains were identified.

From Table 8, it is clear that identity of Sequence 7257 may be stated with a significant level of certainty because the hit score was 6 with an entropy score of 1. However, the same is not true of Sequence 8369, the identity of which ranging from Methylobacterium extorquens to Lactobacillus acidophilus.

Table 10 provides illustration of clustering and tiering based on the phylogenetic tree of a sequence. Here, Enterovirus A and Bovine coronavirus overlap at the order level, “ssRNA positive-strand virsuses' no DNA-stage.” By numbering the tiers, starting from the root (which is defined as being common to all organisms), the distance between the common order of Enterovirus A and Bovine coronavirus is 7 tiers.

Finally, Table 11 provides a result after clustering. In line 4 of Table 11, the order of Enterovirus A and Bovine coronavirus is shown (“ssRNA positive-strand virsuses' no DNA-stage”). The number of branches in the tier is identified as 7 (the number of tiers in the distance between Enterovirus A and Bovine coronavirus.

The methods as described herein provide a novel manner to identifying all known and novel pathogens, vectors, and other genetic material within a specimen that is entirely autonomous. The methods enabling such testing according to the various embodiments here yield extremely and highly complex analysis to be operated on at a low complexity level. Moreover, the embodiments described herein provide computer assisted identification with less personal bias and without impartiality being introduced. The methods are amiable to both cluster and cloud computing, which enables in-house and in-the-field testing, centralizes computer resources, and minimizes labor costs.

Furthermore, embodiments of the present invention may be used as an epidemiological tool by which new and emerging pathogens may be identified. New strains may be quickly identified by sequence and for which assays may be more readily developed.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the scope of the general inventive concept.

TABLE 7

| | | | | | | |
|---|---|---|---|---|---|---|
| Filter Type | Taxon # | Reason for filter | Taxon Name | Commentary Field 1 | Commentary Field 2 | Commentary Field 3 |

#controls

| | | | | | |
|---|---|---|---|---|---|
| filter out | 1246486 | control | Synthetic | Inherited blast name: | Illumina control |
| | | | Enterobacteria | other sequences | sequence |
| | | | [symbol] | | |
| filter out | 10842 | Control | Microvirus | Inherited blast name: | Near relatives of the |
| | | | | viruses | Illumina control |

#suppressed due to frequent observance

| | | | | | | |
|---|---|---|---|---|---|---|
| filter out | 1977402 | commensal_flora | Escherichia | Inherited blast name: | common commensal | |
| | | | [symbol] | [symbol] | [symbol] | |
| filter out | 186765 | commensal_flora | Lambdavirus | Inherited blast name: | common commensal | |
| | | | | [symbol] | [symbol] | |
| filter out | 186789 | commensal_flora | P1virus | Inherited blast name: | common commensal | |
| | | | | [symbol] | [symbol] | |
| filter out | 10662 | commensal_flora | Myoviridae | Genbank common | Inherited blast name: | common commensal |
| | | | | [symbol] | [symbol] | [symbol] |

#metazoa

| | | | | | |
|---|---|---|---|---|---|
| filter out | 33208 | host_metazoa | Metazoa | Genbank common | Inherited blast name: |
| | | | | [symbol] | [symbol] |
| include | 6178 | parasite | Trematoda | Inherited blast name: | |
| | | | | [symbol] | |
| Include | 6199 | Parasite | #Cestoda | Genbank common | Inherited blast name: |
| | | | | [symbol] | [symbol] |
| include | 6231 | parasite | #Nematoda | Genbank common | Inherited blast name: |

#insects & mites & ectoparasites

| | | | | | |
|---|---|---|---|---|---|
| filter out | 6656 | background | Arthropoda | Genbank common | Inherited blast name: |
| include | 121222 | ectoparasite | Pediculus | Inherited blast name: | |
| include | 52282 | ectoparasite | Sarcoptes | Inherited blast name: | |
| include | 121229 | ectoparasite | Pthiridae | Genbank common | Inherited blast name: |
| include | 1658400 | ectoparasite | Hectopsyllidae | Inherited blast name: | |
| | | | | [symbol] | |
| include | 297308 | ectoparasite | Ixodoidea | Inherited blast name: | |
| include | 54283 | ectoparasite | Cuterebrinae | Inherited blast name: | |
| include | 7157 | ectoparasite | Culicidae | Genbank common | Inherited blast name: |
| include | 30079 | ectoparasite | Cimex | Inherited blast name: | |
| include | 27479 | ectoparasite | Reduviidae | Genbank common | Inherited blast name: |
| | | | | [symbol] | [symbol] |
| include | 7205 | ectoparasite | Tabanidae | Genbank common | Inherited blast name: |
| include | 41819 | ectoparasite | Ceratopogonidae | Genbank common | Inherited blast name: |
| include | 27462 | ectoparasite | Austrosimulium | Inherited blast name: | |
| | | | | [symbol] | |
| include | 7197 | Ectoparasite | Psychodidae | Genbank common | Inherited blast name: |

#protozoa parasites & wide eukaryota

| | | | | | |
|---|---|---|---|---|---|
| filter out | 2759 | background | Eukaryota | Genbank common | Inherited blast name: |
| include | 5820 | parasite_protazoa | Plasmodium | Inherited blast name: | |
| include | 5758 | parasite_protazoa | Entamoeba | Inherited blast name: | |
| include | 68459 | parasite_protazoa | Giardiinae | Inherited blast name: | |
| include | 5654 | parasite_protazoa | Trypanosomatida | Inherited blast name: | |
| include | 5810 | parasite_protazoa | Toxoplasma | Inherited blast name: | |
| include | 33677 | parasite_protazoa | Acanthamoebidae | Inherited blast name: | |
| include | 5658 | parasite_protazoa | Leishmania | Inherited blast name: | |
| include | 32594 | parasite_protazoa | Babesiidae | Inherited blast name: | |
| include | 555408 | parasite_protazoa | Balamuthiidae | Inherited blast name: | |
| include | 35082 | parasite_protazoa | Cryptosporidiidae | Inherited blast name: | |
| include | 44417 | parasite_protazoa | Cyclospora | Inherited blast name: | |
| include | 5761 | parasite_protazoa | Naegleria | Inherited blast name: | |
| include | 242060 | parasite_protazoa | Cystoisospora | Inherited blast name: | |

#fungal pathogens

| | | | | | | |
|---|---|---|---|---|---|---|
| filter out | 4751 | background | Fungi | Genbank common | Inherited blast name: | common commensal |
| include | 5475 | pathogen_fungal | Candida | Inherited blast name: | | |
| include | 5052 | pathogen_fungal | Aspergillus | Inherited blast name: | | |
| include | 5415 | pathogen_fungal | Cryptococcus | Inherited blast name: | | |
| include | 5036 | pathogen_fungal | Histoplasma | Inherited blast name: | | |
| include | 4753 | pathogen_fungal | Pneumocystis | Inherited blast name: | | |
| include | 74721 | pathogen_fungal | Stachybotrys | Inherited blast name: | | |
| include | 5550 | pathogen_fungal | Trichophyton | Inherited blast name: | | |
| include | 6029 | pathogen_fungal | Microsporidia | Inherited blast name: | | |
| include | 40354 | pathogen_fungal | Fonsecaea | Inherited blast name: | | |
| include | 100474 | pathogen_fungal | Batrachochytrium | Inherited blast name: | | |
| include | 5500 | pathogen_fungal | Coccidioides | Inherited blast name: | | |
| include | 43987 | pathogen_fungal | Geotrichum | Inherited blast name: | | |
| include | 29907 | pathogen_fungal | Sporothrix | Inherited blast name: | | |
| include | 34390 | pathogen_fungal | Epidermophyton | Inherited blast name: | | |
| include | 91942 | pathogen_fungal | Hortaea | Inherited blast name: | | |
| include | 55193 | pathogen_fungal | Malassezia | Inherited blast name: | | |
| include | 147572 | pathogen_fungal | Piedraia | Inherited blast name: | | |
| include | 40354 | pathogen_fungal | Fonsecaea | Inherited blast name: | | |
| include | 284134 | pathogen_fungal | Sarocladium | Inherited blast name: | | |
| include | 160029 | pathogen_fungal | Neotestudina | Inherited blast name: | | |
| include | 65412 | pathogen_fungal | Phaeoacremoniu | Inherited blast name: | | |
| include | 5596 | pathogen_fungal | Pseudallescheria | Inherited blast name: | | |
| include | 5502 | pathogen_fungal | Curvularia | Inherited blast name: | | |
| include | 82105 | pathogen_fungal | Cladophialophora | Inherited blast name: | | |
| include | 5583 | pathogen_fungal | Exophiala | Inherited blast name: | | |
| include | 703485 | pathogen_fungal | Falciformispora | Inherited blast name: | | |
| include | 100815 | pathogen_fungal | Madurella | Inherited blast name: | | |
| include | 29907 | pathogen_fungal | Pyrenochaeta | Inherited blast name: | | |
| | | | | [symbol] | | |
| include | 34390 | pathogen_fungal | Paracoccidioides | Inherited blast name: | | |
| | | | | [symbol] | | |
| include | 91942 | pathogen_fungal | Entomophthorale | Inherited blast name: | | |
| | | | | [symbol] | | |

#plant/algae pathogens of humans and animals

| | | | | |
|---|---|---|---|---|
| filter out | 33090 | background | Viridiplantae | Inherited blast name: |
| | | | | [symbol] |
| include | 91202 | pathogen_algae | Desmodesmus | Inherited blast name: |
| | | | | [symbol] |
| include | 3110 | pathogen_algae | Prototheca | Inherited blast name: |
| | | | | [symbol] |
| include | 145474 | pathogen_algae | Helicosporidium | Inherited blast name: |
| | | | | [symbol] |

#optional filters: white list for most nasty VIRUS

| | | | | | |
|---|---|---|---|---|---|
| filter out | 10239 | background | Viruses | Inherited blast name: | |
| | | | | [symbol] | |
| include | 10508 | pathogen_virus | Adenoviridae | Inherited blast name: | |
| | | | | [symbol] | |
| include | 464095 | pathogen_virus | Picomavirales | Inherited blast name: | |
| | | | | [symbol] | |
| include | 76804 | pathogen_virus | Nidovariales | Inherited blast name: | |
| | | | | [symbol] | |
| include | 548681 | pathogen_virus | Herpesvirales | Inherited blast name: | |
| | | | | [symbol] | |
| include | 11157 | pathogen_virus | Mononegavirales | Genbank common | |
| | | | | [symbol] | |
| include | 10780 | pathogen_virus | Parvoviridae | Inherited blast name: | |
| | | | | [symbol] | |
| include | 1980410 | pathogen_virus | Bunyavirales | Inherited blast name: | Inherited blast name: |
| | | | | [symbol] | [symbol] |
| include | 10404 | pathogen_virus | Hepadnaviridae | Inherited blast name: | |
| | | | | [symbol] | |
| include | 11050 | pathogen_virus | Flaviviridae | Inherited blast name: | Inherited blast name: |
| | | | | [symbol] | [symbol] |
| include | 39759 | pathogen_virus | Deltavirus | Inherited blast name: | Inherited blast name: |
| | | | | [symbol] | [symbol] |
| include | 11157 | pathogen_virus | Mononegavirales | Inherited blast name: | |
| | | | | [symbol] | |
| include | 151340 | pathogen_virus | Papillomaviridae | Inherited blast name: | Inherited blast name: |
| | | | | [symbol] | [symbol] |
| include | 11308 | pathogen_virus | Orthomyxovirida | Inherited blast name: | Inherited blast name: |
| | | | | [symbol] | [symbol] |
| include | 11617 | pathogen_virus | Arenaviridae | Inherited blast name: | Inherited blast name: |
| | | | | [symbol] | [symbol] |
| include | 10240 | pathogen_virus | Poxviridae | Inherited blast name: | Inherited blast name: |
| | | | | [symbol] | [symbol] |
| include | 11974 | pathogen_virus | Caliciviridae | Inherited blast name: | Inherited blast name: |
| | | | | [symbol] | [symbol] |
| include | 151341 | pathogen_virus | Polyomaviridae | Inherited blast name: | Inherited blast name: |
| | | | | [symbol] | [symbol] |
| include | 10880 | pathogen_virus | Reoviridae | Inherited blast name: | Inherited blast name: |
| | | | | [symbol] | [symbol] |
| include | 11018 | pathogen_virus | Togaviridae | Inherited blast name: | Inherited blast name: |
| | | | | [symbol] | [symbol] |
| include | 11632 | pathogen_virus | Retroviridae | Inherited blast name: | Inherited blast name: |
| | | | | [symbol] | [symbol] |
| include | 39733 | pathogen_virus | Astroviridae | Inherited blast name: | |
| | | | | [symbol] | |

#optional filters; bacteria with a white list for most nasty bacteria
#this list may not be correct for all use cases

| | | | | | | |
|---|---|---|---|---|---|---|
| filter out | 2 | background | Bacteria | Genbank common | Inherited blast name: | Common |
| | | | | [symbol] | [symbol] | [symbol] |
| include | 766 | pathogen_bacteria | Rickettsiales | Genbank common | Inherited blast name: a- | |
| | | | | [symbol] | [symbol] | |
| include | 118969 | pathogen_bacteria | Legionellales | Inherited blast name: g- | | |
| | | | | [symbol] | | |
| include | 1637 | pathogen_bacteria | Listeria | Inherited blast name: | | |
| | | | | [symbol] | | |
| include | 194 | pathogen_bacteria | Campylobacter | Inherited blast name: e- | | |
| | | | | [symbol] | | |
| include | 1279 | pathogen_bacteria | Staphylococcus | Inherited blast name: | | |
| | | | | [symbol] | | |
| include | 543 | pathogen_bacteria | Enterobacteriaceae | Inherited blast name: | | |
| | | | | [symbol] | | |
| include | 138 | pathogen_bacteria | Borrelia | Inherited blast name: | | |
| | | | | [symbol] | | |
| include | 203691 | pathogen_bacteria | Spirochaetes | Inherited blast name: | | |
| | | | | [symbol] | | |
| include | 72293 | pathogen_bacteria | Helicobacteraceae | Inherited blast name: e- | | |
| | | | | [symbol] | | |
| include | 1485 | pathogen_bacteria | Clostridium | Inherited blast name: | | |
| | | | | [symbol] | | |
| include | 662 | pathogen_bacteria | Vibrio | Inherited blast name: g- | | |
| | | | | [symbol] | | |
| include | 773 | pathogen_bacteria | Bartonella | Inherited blast name: a- | | |
| include | 1301 | pathogen_bacteria | Streptococcus | Inherited blast name: | | |
| filter out | 204429 | pathogen_bacteria | Chlamydia | Inherited blast name: | | |
| include | 1716 | pathogen_bacteria | Corynebacterium | Inherited blast name: | | |
| include | 85007 | pathogen_bacteria | Corynebacterium | Inherited blast name: | | |
| | | | | [symbol] | | |
| include | 1350 | pathogen_bacteria | Corynebacterium | Inherited blast name: | | |
| | | | | [symbol] | | |
| include | 468 | pathogen_bacteria | Enterococcus | Inherited blast name: | | |
| | | | | [symbol] | | |
| include | 28263 | pathogen_bacteria | Moraxellaceae | Inherited blast name: g- | | |
| include | 86661 | pathogen_bacteria | Arcanobacterium | Inherited blast name: | | |
| include | 1654 | pathogen_bacteria | Bacillus cereus | Inherited blast name: | | |
| include | 1743 | pathogen_bacteria | Actinomyces | Inherited blast name: | | |
| include | 286 | pathogen_bacteria | Propionibacterium | Inherited blast name: | | |
| | | | | [symbol] | | |
| include | 816 | pathogen_bacteria | Pseudomonas | Inherited blast name: | | |
| include | 118882 | pathogen_bacteria | Brucellaceae | Inherited blast name: a- | | |
| include | 119060 | pathogen_bacteria | Burkholderiaceae | Inherited blast name: b- | | |
| | | | | [symbol] | | |
| include | 194 | pathogen_bacteria | Campylobacter | Inherited blast name: e- | | |
| | | | | [symbol] | | |
| include | 724 | pathogen_bacteria | Haemophilus | Inherited blast name: gr- | | |
| | | | | [symbol] | | |
| filter out | 203492 | pathogen_bacteria | Fusobacteriaceae | Inherited blast name: | | |
| | | | | [symbol] | | |
| include | 482 | pathogen_bacteria | Neisseria | Inherited blast name: b- | | |
| | | | | [symbol] | | |
| include | 32257 | pathogen_bacteria | Kingella | Inherited blast name: b- | | |
| | | | | [symbol] | | |
| include | 517 | pathogen_bacteria | Bordetella | Inherited blast name: b- | | |
| | | | | [symbol] | | |
| include | 629 | pathogen_bacteria | Yersinia | Inherited blast name: | | |
| | | | | [symbol] | | |
| include | 34064 | pathogen_bacteria | Francisellaceae | Inherited blast name: g- | | |
| | | | | [symbol] | | |
| include | 2092 | pathogen_bacteria | Mycoplasmataceae | Inherited blast name: | | |
| | | | | [symbol] | | |
| include | 838 | pathogen_bacteria | Prevotella | Inherited blast name: | | |
| | | | | [symbol] | | |
| include | 620 | pathogen_bacteria | Shigella | Inherited blast name: | | |
| | | | | [symbol] | | |

[symbol]

indicates data missing or illegible when filed

TABLE 8

| Entropy | Hit | | | Taxon | Max | % |
|---|---|---|---|---|---|---|
| Score | Score | Database ID | Database ID | code | score | ID |
| =1 | =6 | @trn_7257 = 6 | gi\|15081544\|ref\|NC_003045.1\| | 11128 | 209 | 95.42 |
| | | @trn_8369 = 1 | gi\|1140783874\|ref\|NZ_CP019322.1\| | 408 | 327 | 98.91 |
| | | @trn_8369 = 1 | gi\|1140783874\|ref\|NZ_CP019322.1\| | 408 | 327 | 98.91 |
| =6 | +5 | @trn_8369 = 1 | gi\|1140783874\|ref\|NZ_CP019322.1\| | 408 | 327 | 98.91 |
| | | @trn_8369 = 1 | gi\|1140783874\|ref\|NZ_CP019322.1\| | 408 | 327 | 98.91 |
| | | @trn_8369 = 1 | gi\|1140783874\|ref\|NZ_CP019322.1\| | 408 | 327 | 98.91 |
| +2 | +2 | @trn_8369 = 1 | gi\|829077173\|ref\|NZ_LCZM01000045.1\| | 1076 | 302 | 96.7 |
| | | @trn_8369 = 1 | gi\|764536604\|ref\|NZ_JXXE01000256.1\| | 1076 | 291 | 96.09 |
| +1 | +1 | @trn_8369 = 1 | gi\|1121310174\|ref\|NZ_LKUS01000062.1\| | 1770 | 327 | 98.91 |
| +1 | +1 | @trn_8369 = 1 | gi\|1140877006\|ref\|NZ_LACA01000120.1\| | 31998 | 327 | 98.91 |
| +2 | +2 | @trn_8369 = 1 | gi\|944512679\|ref\|NZ_LMAR01000067.1\| | 53254 | 296 | 96.15 |
| | | @trn_8369 = 1 | gi\|1160733327\|ref\|NZ_FUYX01000002.1\| | 53254 | 296 | 9615 |
| +1 | +1 | @trn_8369 = 1 | gi\|926285648\|ref\|NZ_LGEJ01000021.1\| | 53367 | 327 | 98.91 |
| +1 | +1 | @trn_8369 = 1 | gi\|926273650\|ref\|NZ_LGE101000052.1\| | 68259 | 361 | 98.09 |
| | | @trn_8369 = 1 | gi\|484101441\|ref\|NZ_BACT01000737.1\| | 91459 | 361 | 98.09 |
| +1 | +1 | @trn_8369 = 1 | gi\|484134505\|ref\|NZ_BADE01000276.1\| | 95563 | 327 | 98.91 |
| +1 | +1 | @trn_8369 = 1 | gi\|821189942\|ref\|NZ_LBIA01000001.1\| | 211460 | 291 | 96.09 |
| +1 | +1 | @trn_8369 = 1 | gi\|1028641727\|ref\|NZ_LSNC01000079.1\| | 223967 | 327 | 98.91 |
| +4 | +14 | @trn_8369 = 1 | gi\|985611191\|ref\|NZ_AP014705.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP0147.04.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|985611990\|ref\|NZ_AP014704.1\| | 270351 | 316 | 97.83 |
| | | @trn_8369 = 1 | gi\|969894647\|ref\|NZ_LDRM01000027.1\| | 270351 | 311 | 97.28 |
| | | @trn_8369 = 1 | gi\|969893888\|ref\|NZ_LDRL01000092.1\| | 270351 | 311 | 97.28 |
| | | @trn_8369 = 1 | gi\|860569244\|ref\|NZ_LABX01000097.1\| | 270351 | 311 | 97.28 |
| +1 | +5 | @trn_8369 = 1 | gi\|240136783\|ref\|NC_012808.1\| | 272630 | 327 | 98.91 |
| | | @trn_8369 = 1 | gi\|240136783\|ref\|NC_012808.1\| | 272630 | 327 | 98.91 |
| | | @trn_8369 = 1 | gi\|240136783\|ref\|NC_012808.1\| | 272630 | 327 | 98.91 |
| | | @trn_8369 = 1 | gi\|240136783\|ref\|NC_012808.1\| | 272630 | 327 | 98.91 |
| | | @trn_8369 = 1 | gi\|240136783\|ref\|NC_012808.1\| | 272630 | 327 | 98.91 |
| +1 | +1 | @trn_8369 = 1 | gi\|860512790\|ref\|NZ_LABY01000145.1\| | 298794 | 311 | 97.28 |
| +1 | +2 | @trn_8369 = 1 | gi\|91974482\|ref\|NC_007958.1\| | 316057 | 291 | 96.09 |
| | | @trn_8369 = 1 | gi\|91974482\|ref\|NC_007958.1\| | 316057 | 291 | 96.09 |
| +1 | +1 | @trn_8369 = 1 | gi\|86747127\|ref\|NC_007778.1\| | 316058 | 291 | 96.09 |
| +1 | +1 | @trn_8369 = 1 | gi\|482991224\|ref\|NZ_KB900609.1\| | 398261 | 311 | 97.28 |
| | | @trn_8369 = 1 | gi\|482991224\|ref\|NZ_KB900609.1\| | 398261 | 311 | 97.28 |
| | | @trn_8369 = 1 | gi\|482991224\|ref\|NZ_KB900609.1\| | 398261 | 311 | 97.28 |
| | | @trn_8369 = 1 | gi\|482991224\|ref\|NZ_KB900609.1\| | 398261 | 311 | 97.28 |
| | | @trn_8369 = 1 | gi\|482991224\|ref\|NZ_KB900609.1\| | 398261 | 311 | 97.28 |
| | | @trn_8369 = 1 | gi\|482991224\|ref\|NZ_KB900609.1\| | 398261 | 311 | 97.28 |
| +1 | +4 | @trn_8369 = 1 | gi\|1129420732\|ref\|NZ_CP015367.1\| | 482323 | 361 | 98.09 |
| | | @trn_8369 = 1 | gi\|1129420732\|ref\|NZ_CP015367.1\| | 482323 | 361 | 98.09 |
| | | @trn_8369 = 1 | gi\|1129420732\|ref\|NZ_CP015367.1\| | 482323 | 361 | 98.09 |
| | | @trn_8369 = 1 | gi\|1129420732\|ref\|NZ_CP015367.1\| | 482323 | 361 | 98.09 |
| +1 | +5 | @trn_8369 = 1 | gi\|163849457\|ref\|NC_010172.1\| | 419610 | 327 | 98.91 |
| | | @trn_8369 = 1 | gi\|163849457\|ref\|NC_010172.1\| | 419610 | 327 | 98.91 |
| | | @trn_8369 = 1 | gi\|163849457\|ref\|NC_010172.1\| | 419610 | 327 | 98.91 |
| | | @trn_8369 = 1 | gi\|163849457\|ref\|NC_010172.1\| | 419610 | 327 | 98.91 |
| | | @trn_8369 = 1 | gi\|163849457\|ref\|NC_010172.1\| | 419610 | 327 | 98.91 |
| +1 | +6 | @trn_8369 = 1 | gi\|170738367\|ref\|NC_010511.1\| | 426117 | 311 | 97.28 |
| | | @trn_8369 = 1 | gi\|170738367\|ref\|NC_010511.1\| | 426117 | 311 | 97.28 |
| | | @trn_8369 = 1 | gi\|170738367\|ref\|NC_010511.1\| | 426117 | 311 | 97.28 |
| | | @trn_8369 = 1 | gi\|170738367\|ref\|NC_010511.1\| | 426117 | 311 | 97.28 |
| | | @trn_8369 = 1 | gi\|170738367\|ref\|NC_010511.1\| | 426117 | 311 | 97.28 |
| | | @trn_8369 = 1 | gi\|170738367\|ref\|NC_010511.1\| | 426117 | 305 | 96.76 |
| +1 | +6 | @trn_8369 = 1 | gi\|170745058\|ref\|NC_010510.1\| | 426355 | 327 | 98.91 |
| | | @trn_8369 = 1 | gi\|170745058\|ref\|NC_010510.1\| | 426355 | 327 | 98.91 |
| | | @trn_8369 = 1 | gi\|170745058\|ref\|NC_010510.1\| | 426355 | 327 | 98.91 |
| | | @trn_8369 = 1 | gi\|170745058\|ref\|NC_010510.1\| | 426355 | 327 | 98.91 |
| | | @trn_8369 = 1 | gi\|170745058\|ref\|NC_010510.1\| | 426355 | 327 | 98.91 |
| | | @trn_8369 = 1 | gi\|170745058\|ref\|NC_010510.1\| | 426355 | 327 | 98.91 |
| +3 | +3 | @trn_8369 = 1 | gi\|1034535815\|ref\|NZ_LWHQ01000093.1\| | 427683 | 311 | 97.28 |
| | | @trn_8369 = 1 | gi\|860551095\|ref\|NZ_JTHG01000052.1\| | 427683 | 311 | 97.28 |
| | | @trn_8369 = 1 | gi\|860466786\|ref\|NZ_JTHF01000318.1\| | 427683 | 311 | 97.28 |
| +1 | +5 | @trn_8369 = 1 | gi\|218528082\|ref\|NC_011757.1\| | 440085 | 327 | 98.91 |
| | | @trn_8369 = 1 | gi\|218528082\|ref\|NC_011757.1\| | 440085 | 327 | 98.91 |
| | | @trn_8369 = 1 | gi\|218528082\|ref\|NC_011757.1\| | 440085 | 327 | 98.91 |
| | | @trn_8369 = 1 | gi\|218528082\|ref\|NC_011757.1\| | 440085 | 327 | 98.91 |
| | | @trn_8369 = 1 | gi\|218528082\|ref\|NC_011757.1\| | 440085 | 327 | 98.91 |
| +1 | +5 | @trn_8369 = 1 | gi\|188579286\|ref\|NC_010725.1\| | 441620 | 327 | 98.1 |
| | | @trn_8369 = 1 | gi\|188579286\|ref\|NC_010725.1\| | 441620 | 327 | 98.1 |
| | | @trn_8369 = 1 | gi\|188579286\|ref\|NC_010725.1\| | 441620 | 327 | 98.1 |
| | | @trn_8369 = 1 | gi\|188579286\|ref\|NC_010725.1\| | 441620 | 327 | 98.1 |
| | | @trn_8369 = 1 | gi\|188579286\|ref\|NC_010725.1\| | 441620 | 327 | 98.1 |
| +1 | +7 | @trn_8369 = 1 | gi\|22920054\|ref\|NC_011894.1\| | 460265 | 305 | 97.25 |
| | | @trn_8369 = 1 | gi\|22920054\|ref\|NC_011894.1\| | 460265 | 305 | 97.25 |
| | | @trn_8369 = 1 | gi\|22920054\|ref\|NC_011894.1\| | 460265 | 305 | 97.25 |
| | | @trn_8369 = 1 | gi\|22920054\|ref\|NC_011894.1\| | 460265 | 305 | 97.25 |
| | | @trn_8369 = 1 | gi\|22920054\|ref\|NC_011894.1\| | 460265 | 305 | 97.25 |
| | | @trn_8369 = 1 | gi\|22920054\|ref\|NC_011894.1\| | 460265 | 305 | 97.25 |
| | | @trn_8369 = 1 | gi\|22920054\|ref\|NC_011894.1\| | 460265 | 305 | 97.25 |
| +1 | +1 | @trn_8369 = 1 | gi\|483993734\|ref\|NZ_AMXU01000096.1\| | 648885 | 327 | 98.91 |
| +1 | +2 | @trn_8369 = 1 | gi\|316931396\|ref\|NC_014834.1\| | 652103 | 302 | 96.7 |
| | | @trn_8369 = 1 | gi\|316931396\|ref\|NC_014834.1\| | 652103 | 302 | 96.7 |
| +1 | +5 | @trn_8369 = 1 | gi\|254558653\|ref\|NC_012988.1\| | 661410 | 327 | 98.91 |
| | | @trn_8369 = 1 | gi\|254558653\|ref\|NC_012988.1\| | 661410 | 327 | 98.91 |
| | | @trn_8369 = 1 | gi\|254558653\|ref\|NC_012988.1\| | 661410 | 327 | 98.91 |
| | | @trn_8369 = 1 | gi\|254558653\|ref\|NC_012988.1\| | 661410 | 327 | 98.91 |
| | | @trn_8369 = 1 | gi\|254558653\|ref\|NC_012988.1\| | 661410 | 327 | 98.91 |
| +1 | +1 | @trn_8369 = 1 | gi\|389691362\|ref\|NZ_JH660642.1\| | 864069 | 302 | 96.7 |
| +1 | +1 | @trn_8369 = 1 | gi\|418061099\|ref\|NZ_AGJK01000112.1\| | 882800 | 327 | 98.91 |
| +1 | +1 | @trn_8369 = 1 | gi\|448879098\|ref\|NZ_KB375282.1\| | 883078 | 291 | 96.09 |
| +1 | +1 | @trn_8369 = 1 | gi\|475651767\|ref\|NZ_ANPA01000016.1\| | 908290 | 327 | 98.91 |
| +1 | +5 | @trn_8369 = 1 | gi\|984669198\|ref\|NZ_CP006992.1\| | 925818 | 327 | 98.91 |
| | | @trn_8369 = 1 | gi\|984669198\|ref\|NZ_CP006992.1\| | 925818 | 327 | 98.91 |
| | | @trn_8369 = 1 | gi\|984669198\|ref\|NZ_CP006992.1\| | 925818 | 327 | 98.91 |
| | | @trn_8369 = 1 | gi\|984669198\|ref\|NZ_CP006992.1\| | 925818 | 327 | 98.91 |
| | | @trn_8369 = 1 | gi\|984669198\|ref\|NZ_CP006992.1\| | 925818 | 327 | 98.91 |
| +1 | +2 | @trn_8369 = 1 | gi\|1057378984\|ref\|NZ_LVYV01000001.1\| | 943830 | 291 | 96.09 |
| | | @trn_8369 = 1 | gi\|1057378984\|ref\|NZ_LVYV01000001.1\| | 943830 | 291 | 96.09 |
| +2 | +2 | @trn_8369 = 1 | gi\|821562761\|ref\|NZ_LN811386.1\| | 1033741 | 302 | 96.7 |
| | | @trn_8369 = 1 | gi\|880988436\|ref\|NZ_CAHM010000373.1\| | 1033741 | 302 | 96.7 |
| +1 | +1 | @trn_8369 = 1 | gi\|393766792\|ref\|NZ_AKFK01000054.1\| | 1096546 | 339 | 96.17 |
| +1 | +5 | @trn_8369 = 1 | gi\|652920628\|ref\|NZ_K1912577.1\| | 1101191 | 302 | 96.7 |
| | | @trn_8369 = 1 | gi\|652920628\|ref\|NZ_K1912577.1\| | 1101191 | 302 | 96.7 |
| | | @trn_8369 = 1 | gi\|652920628\|ref\|NZ_K1912577.1\| | 1101191 | 302 | 96.7 |
| | | @trn_8369 = 1 | gi\|652920628\|ref\|NZ_K1912577.1\| | 1101191 | 302 | 96.7 |
| | | @trn_8369 = 1 | gi\|652920628\|ref\|NZ_K1912577.1\| | 1101191 | 302 | 96.7 |
| +1 | +5 | @trn_8369 = 1 | gi\|486345215\|ref\|NZ_KB910516.1\| | 1101192 | 302 | 96.7 |
| | | @trn_8369 = 1 | gi\|486345215\|ref\|NZ_KB910516.1\| | 1101192 | 302 | 96.7 |
| | | @trn_8369 = 1 | gi\|486345215\|ref\|NZ_KB910516.1\| | 1101192 | 302 | 96.7 |
| | | @trn_8369 = 1 | gi\|486345215\|ref\|NZ_KB910516.1\| | 1101192 | 302 | 96.7 |
| +1 | +1 | @trn_8369 = 1 | gi\|487380982\|ref\|NZ_KB911351.1\| | 1172187 | 327 | 98.91 |
| +1 | +1 | @trn_8369 = 1 | gi\|589884799\|ref\|NZ_HG326655.1\| | 1197906 | 291 | 96.09 |
| +1 | +1 | @trn_8369 = 1 | gi\|827107632\|ref\|NZ_LCYG01000082.1\| | 1225564 | 302 | 96.7 |
| +1 | +1 | @trn_8369 = 1 | gi\|639246717\|ref\|NZ_APHQ01000008.1\| | 1293051 | 291 | 96.09 |
| +1 | +1 | @trn_8369 = 1 | gi\|860483090\|ref\|NZ_JX0D01000035.1\| | 1295136 | 311 | 97.28 |
| +1 | +1 | @trn_8369 = 1 | gi\|1639257501\|ref\|NZ_APJ101000006.1\| | 1297860 | 291 | 96.09 |
| +1 | +1 | @trn_8369 = 1 | gi\|639259540\|ref\|NZ_APJH01000012.1\| | 1297861 | 291 | 96.09 |
| +1 | +5 | @trn_8369 = 1 | gi\|639260636\|ref\|NZ_APJG01000003.1\| | 1297862 | 291 | 96.09 |
| +1 | +1 | @trn_8369 = 1 | gi\|639262581\|ref\|NZ_APJF01000010.1\| | 1297863 | 291 | 96.09 |
| +1 | +1 | @trn_8369 = 1 | gi\|629264774\|ref\|NZ_1297864.1\| | 1297864 | 291 | 96.09 |
| +1 | +1 | @trn_8369 = 1 | gi\|640487958\|ref\|NZ_AVBK01000004.1\| | 1320552 | 291 | 96.09 |
| +1 | +1 | @trn_8369 = 1 | gi\|640488112\|ref\|NZ_AVBL01000011.1\| | 1320553 | 291 | 96.09 |
| +1 | +1 | @trn_8369 = 1 | gi\|640479677\|ref\|NZ_AVBM01000004.1 | 1320554 | 291 | 96.09 |
| +1 | +1 | @trn_8369 = 1 | gi\|653066036\|ref\|NZ_JAEA01000027.1\| | 1336243 | 302 | 96.7 |
| +1 | +1 | @trn_8369 = 1 | gi\|657881342\|ref\|NZ_JN1J01000042.1\| | 1380355 | 291 | 96.09 |
| +1 | +1 | @trn_8369 = 1 | gi\|739157246\|ref\|NZ_JQNH01000001.1\| | 1411123 | 307 | 97.25 |
| +1 | +1 | @trn_8369 = 1 | gi\|658816309\|ref\|NZ_AYUB01000055.1\| | 1421011 | 291 | 96.09 |
| +1 | +4 | @trn_8369 = 1 | gi\|1094003594\|ref\|NZ_CP017640.1\| | 1479019 | 327 | 98.91 |
| | | @trn_8369 = 1 | gi\|1094003594\|ref\|NZ_CP017640.1 | 1479019 | 327 | 98.91 |
| | | @trn_8369 = 1 | gi\|1094003594\|ref\|NZ_CP017640.1 | 1479019 | 327 | 98.91 |
| | | @trn_8369 = 1 | gi\|1094003594\|ref\|NZ_CP017640.1 | 1479019 | 327 | 98.91 |
| +1 | +1 | @trn_8369 = 1 | gi\|930063430\|ref\|NZ_LIC01000108.1\| | 1523430 | 291 | 96.09 |
| +1 | +1 | @trn_8369 = 1 | gi\|914809853\|ref\|NZ_LHCD01000108.1\| | 1692501 | 339 | 96.17 |
| +1 | +1 | @trn_8369 = 1 | gi\|959937952\|ref\|NZ_LKK001000100.1\| | 1730094 | 339 | 96.17 |
| +1 | +1 | @trn_8369 = 1 | gi\|947793680\|ref\|NZ_LMMG01000030.1\| | 1736242 | 302 | 96.7 |
| +1 | +1 | @trn_8369 = 1 | gi\|947605418\|ref\|NZ_LMMI01000001.1\| | 1736243 | 302 | 96.7 |
| +1 | +1 | @trn_8369 = 1 | gi\|947615570\|ref\|NZ_LMMK01000040.1\| | 1736244 | 302 | 96.7 |
| +1 | +1 | @trn_8369 = 1 | gi\|947693279\|ref\|NZ_LMML01000021.1\| | 1736245 | 302 | 96.7 |
| +1 | +1 | @trn_8369 = 1 | gi\|947803454\|ref\|NZ_LMMN01000003.1\| | 1736246 | 327 | 98.91 |
| +1 | +1 | @trn_8369 = 1 | gi\|947773098\|ref\|NZ_LMMP01000052.1\| | 1736247 | 302 | 96.7 |
| +1 | +1 | @trn_8369 = 1 | gi\|947492327\|ref\|NZ_LMMQ01000036.1\| | 1736248 | 327 | 98.91 |
| +1 | +1 | @trn_8369 = 1 | gi\|947559798\|ref\|NZ_LMRM01000023.1\| | 173620 | 302 | 96.7 |
| +1 | +1 | @trn_8369 = 1 | gi\|947432928\|ref\|NZ_LMMU01000001.1\| | 1736251 | 333 | 95.69 |
| +1 | +1 | @trn_8369 = 1 | gi\|947644021\|ref\|NZ_LMMW01000012.1\| | 1736252 | 302 | 96.7 |
| +1 | +1 | @trn_8369 = 1 | gi\|647701314\|ref\|NZ_LMMX01000034.1\| | 1736253 | 302 | 96.7 |
| +1 | +1 | @trn_8369 = 1 | gi\|947816984\|ref\|NZ_LMMZ01000037.1\| | 1736254 | 302 | 96.7 |
| +1 | +1 | @trn_8369 = 1 | gi\|947624330\|ref\|NZ_LMND01000012.1\| | 1736256 | 361 | 98.09 |
| +1 | +1 | @trn_8369 = 1 | gi\|947836849\|ref\|NZ_LMNE01000045.1\| | 1736257 | 302 | 96.7 |
| +1 | +1 | @trn_8369 = 1 | gi\|947513087\|ref\|NZ_LMNG01000012.1\| | 1736258 | 302 | 96.7 |
| +1 | +1 | @trn_8369 = 1 | gi\|947527031\|ref\|NZ_LMNJ01000045.1\| | 1736259 | 302 | 96.7 |
| +1 | +1 | @trn_8369 = 1 | gi\|947827736\|ref\|NZ_LMNL01000036.1\| | 1736260 | 302 | 96.7 |
| +1 | +1 | @trn_8369 = 1 | gi\|947616289\|ref\|NZ_LMNN01000014.1\| | 1736261 | 327 | 98.91 |
| +1 | +1 | @trn_8369 = 1 | gi\|947846816\|ref\|NZ_LMNP01000018.1\| | 1736262 | 327 | 98.91 |
| +1 | +1 | @trn_8369 = 1 | gi\|9474546412\|ref\|NZ_LMNQ01000001.1\| | 1736263 | 327 | 98.91 |
| +1 | +1 | @trn_8369 = 1 | gi\|947541665\|ref\|NZ_LMNS01000034.1\| | 1736264 | 327 | 98.91 |
| +1 | +1 | @trn_8369 = 1 | gi\|9471883811\|ref\|NZ_LMNU01000023.1\| | 1736265 | 302 | 96.7 |
| +1 | +1 | @trn_8369 = 1 | gi\|948036732\|ref\|NZ_LMRN0100002.1\| | 1736300 | 302 | 96.7 |
| +1 | +1 | @trn_8369 = 1 | gi\|94787446\|ref\|NZ_LMPY01000078.1\| | 1736352 | 327 | 98.4 |
| +1 | +1 | @trn_8369 = 1 | gi\|946968425\|ref\|NZ_LMQK01000012.1\| | 1736364 | 361 | 98.09 |
| +1 | +1 | @trn_8369 = 1 | gi\|947586856\|ref\|NZ_LMQV01000041.1\| | 1736382 | 316 | 97.83 |
| +1 | +1 | @trn_8369 = 1 | gi\|947721136\|ref\|NZ_LMRA01000045.1\| | 1736385 | 302 | 96.7 |
| +1 | +1 | @trn_8369 = 1 | gi\|947749269\|ref\|NZ_LMND01000012.1\| | 1736386 | 361 | 98.09 |
| +1 | +1 | @trn_8369 = 1 | gi\|947836843\|ref\|NZ_LMRC01000045.1\| | 1736387 | 302 | 96.7 |
| +1 | +1 | @trn_8369 = 1 | gi\|947639327\|ref\|NZ_LMDP01000003.1\| | 1736436 | 291 | 96.09 |
| +1 | +1 | @trn_8369 = 1 | gi\|1011023503\|ref\|NZ_LSIM01000122.1\| | 1768759 | 291 | 96.09 |
| +1 | +1 | @trn_8369 = 1 | gi\|1011405890\|ref\|NZ_LSIN01000075.1\| | 1768760 | 291 | 96.09 |
| +1 | +1 | @trn_8369 = 1 | gi\|947846816\|ref\|NZ_LSIX01000712.1\| | 1768765 | 324 | 97.4 |
| +1 | +5 | @trn_8369 = 1 | gi\|1189846260\|ref\|NZ_CP021054.1\| | N/A | 327 | 98.91 |
| | | @trn_8369 = 1 | gi\|1189846260\|ref\|NZ_CP021054.1\| | N/A | 327 | 98.91 |
| | | @trn_8369 = 1 | gi\|1189846260\|ref\|NZ_CP021054.1\| | N/A | 327 | 98.91 |
| | | @trn_8369 = 1 | gi\|1189846260\|ref\|NZ_CP021054.1\| | N/A | 327 | 98.91 |
| | | @trn_8369 = 1 | gi\|1189846260\|ref\|NZ_CP021054.1\| | N/A | 327 | 98.91 |
| +1 | +2 | @trn_10063 = 2 | gi\|1125843910\|ref\|NZ_MSIF01000054.1 | 485602 | 313 | 96.37 |
| +1 | +2 | @trn_10063 = 2 | gi\|1053280538\|ref\|NZ_MCRG01000108.1 | 53346 | 313 | 96.37 |
| +1 | +2 | @trn_10063 = 2 | gi\|1027691334\|ref\|NZ_LSBT01000070.1 | 562 | 313 | 96.37 |
| +1 | +2 | @trn_10063 = 2 | gi\|29366675\|ref\|NC_000866.4 | 10665 | 313 | 96.37 |
| +1 | +2 | @trn_10063 = 2 | gi\|1167963571\|ref\|NZ_MXSV01000119.1 | 611 | 302 | 95.34 |
| +1 | +2 | @trn_10063 = 2 | gi\|1167890983\|ref\|NZ_MXST01000001.1 | 98360 | 302 | 95.34 |
| +1 | +2 | @trn_10063 = 2 | gi\|953357764\|ref\|NC_028448.1 | 1720504 | 302 | 95.34 |
| +1 | +2 | @trn_10063 = 2 | gi\|116326222\|ref\|NC_008515.1 | 45406 | 298 | 95.74 |

| Entropy | Hit | | |
|---|---|---|---|
| Score | Score | Database ID | Name |
| =1 | =6 | @trn_7257 = 6 | Bovine coronavirus, complete genome |
| | | @trn_8369 = 1 | Methylobacterium extorquens strain PSBB040, complete genome |
| | | @trn_8369 = 1 | Methylobacterium extorquens strain PSBB040, complete genome |
| =6 | +5 | @trn_8369 = 1 | Methylobacterium extorquens strain PSBB040, complete genome |
| | | @trn_8369 = 1 | Methylobacterium extorquens strain PSBB040, complete genome |
| | | @trn_8369 = 1 | Methylobacterium extorquens strain PSBB040, complete genome |
| +2 | +2 | @trn_8369 = 1 | Rhodopseudomonas palustris strain 42OL conntig45, |
| | | | whole genome shotgun sequence |
| | | @trn_8369 = 1 | Rhodopseudomonas palustris strain BAL398 c293\|2759c662.853943, |
| | | | whole genome shotgun sequence |
| +1 | +1 | @trn_8369 = 1 | Mycobacterium avium subsp. paratuberculosis strain 2015WD-1 |
| | | | contig_62, whole genome shotgun sequence |
| +1 | +1 | @trn_8369 = 1 | Methylobacterium radiotolerans strain RE1.2 contig_120, |
| | | | whole genome shotgun sequence |
| +2 | +2 | @trn_8369 = 1 | Bosea thiooxidans strain CGMCC 9174 V5-&, |
| | | | whole genome shotgun sequence |
| | | @trn_8369 = 1 | Bosea thiooxidans strain DSM 9563, |
| | | | whole genome shotgun sequence |
| +1 | +1 | @trn_8369 = 1 | Asanoa ferruginea strain NRRL B-16430 P073contig 116.1, |
| | | | whole genome shotgun sequence |
| +1 | +1 | @trn_8369 = 1 | Streptomyces purpurogeneiscleroticus strain NRRL B-2952 |
| | | | P066contig145.1, whole genome shotgun sequence |
| | | @trn_8369 = 1 | Methylobacterium sp. B2, whole genome shotgun sequence |
| +1 | +1 | @trn_8369 = 1 | Methylobacterium sp. B34, whole genome shotgun sequence |
| +1 | +1 | @trn_8369 = 1 | Afipia massiliensis strain LC387 LC387_contig1, |
| | | | whole genome shotgun |
| +1 | +1 | @trn_8369 = 1 | Methylobacterium populi strain CD11_7 CD11_7_contig1, |
| | | | whole genome shotgun |
| +4 | +14 | @trn_8369 = 1 | Methylobacterium aquaticum plasmid pMaq22A-1p DNA, |
| | | | complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum DNA, complete genome, strain MA-22A |
| | | @trn_8369 = 1 | Methylobacterium aquaticum strain NS229 contig_27, |
| | | | whole genome shotgun sequence |
| | | @trn_8369 = 1 | Methylobacterium aquaticum strain NS228 contig_92, , |
| | | | whole genome shotgun sequence |
| | | @trn_8369 = 1 | Methylobacterium aquaticum strain DSM 16371 contig_97, , |
| | | | whole genome shotgun sequence |
| +1 | +5 | @trn_8369 = 1 | Methylobacterium extorquens AM1, complete genome |
| | | @trn_8369 = 1 | Methylobacterium extorquens AM1, complete genome |
| | | @trn_8369 = 1 | Methylobacterium extorquens AM1, complete genome |
| | | @trn_8369 = 1 | Methylobacterium extorquens AM1, complete genome |
| | | @trn_8369 = 1 | Methylobacterium extorquens M1, complete genome |
| +1 | +1 | @trn_8369 = 1 | Methylobacterium variable strain DSM 16961 contig 145, |
| | | | whole genome shotgun sequence |
| +1 | +2 | @trn_8369 = 1 | Rhodopseudomonas palustris BisB5, complete genome |
| | | @trn_8369 = 1 | Rhodopseudomonas palustris BisB5, complete genome |
| +1 | +1 | @trn_8369 = 1 | Rhodopseudomonas palustris HaA2, complete genome |
| +1 | +1 | @trn_8369 = 1 | Methylobacterium sp. WSM2598 MET2598DRAFT_scaffold1.1, |
| | | | whole genome shotgun sequence |
| | | @trn_8369 = 1 | Methylobacterium sp. WSM2598 MET2598DRAFT_scaffold1.1, |
| | | | whole genome shotgun sequence |
| | | @trn_8369 = 1 | Methylobacterium sp. WSM2598 MET2598DRAFT_scaffold1.1, |
| | | | whole genome shotgun sequence |
| | | @trn_8369 = 1 | Methylobacterium sp. WSM2598 MET2598DRAFT_scaffold1.1, |
| | | | whole genome shotgun sequence |
| | | @trn_8369 = 1 | Methylobacterium sp. WSM2598 MET2598DRAFT_scaffold1.1, |
| | | | whole genome shotgun sequence |
| | | @trn_8369 = 1 | Methylobacterium sp. WSM2598 MET2598DRAFT_scaffold1.1, |
| | | | whole genome shotgun sequence |
| +1 | +4 | @trn_8369 = 1 | Methylobacterium phyllosphaerae strain CBMB27, complete genome |
| | | @trn_8369 = 1 | Methylobacterium phyllosphaerae strain CBMB27, complete genome |
| | | @trn_8369 = 1 | Methylobacterium phyllosphaerae strain CBMB27, complete genome |
| | | @trn_8369 = 1 | Methylobacterium phyllosphaerae strain CBMB27, complete genome |
| +1 | +5 | @trn_8369 = 1 | Methylobacterium extorquens PA1, complete genome |
| | | @trn_8369 = 1 | Methylobacterium extorquens PA1, complete genome |
| | | @trn_8369 = 1 | Methylobacterium extorquens PA1, complete genome |
| | | @trn_8369 = 1 | Methylobacterium extorquens PA1, complete genome |
| | | @trn_8369 = 1 | Methylobacterium extorquens PA1, complete genome |
| +1 | +6 | @trn_8369 = 1 | Methylobacterium sp. 4-46, complete genome |
| | | @trn_8369 = 1 | Methylobacterium sp. 4-46, complete genome |
| | | @trn_8369 = 1 | Methylobacterium sp. 4-46, complete genome |
| | | @trn_8369 = 1 | Methylobacterium sp. 4-46, complete genome |
| | | @trn_8369 = 1 | Methylobacterium sp. 4-46, complete genome |
| | | @trn_8369 = 1 | Methylobacterium sp. 4-46, complete genome |
| +1 | +6 | @trn_8369 = 1 | Methylobacterium radiotolerans JCM 2831 plasmid pMRAD01, |
| | | | complete sequence |
| | | @trn_8369 = 1 | Methylobacterium radiotolerans JCM 2831 plasmid pMRAD01, |
| | | | complete sequence |
| | | @trn_8369 = 1 | Methylobacterium radiotolerans JCM 2831 plasmid pMRAD01, |
| | | | complete sequence |
| | | @trn_8369 = 1 | Methylobacterium radiotolerans JCM 2831 plasmid pMRAD01, |
| | | | complete sequence |
| | | @trn_8369 = 1 | Methylobacterium radiotolerans JCM 2831 plasmid pMRAD01, |
| | | | complete sequence |
| | | @trn_8369 = 1 | Methylobacterium radiotolerans JCM 2831 plasmid pMRAD01, |
| | | | complete sequence |
| +3 | +3 | @trn_8369 = 1 | Methylobacterium platani strain PMB02 contig093, |
| | | | whole genome shotgun sequence |
| | | @trn_8369 = 1 | Methylobacterium platani strain PMB02 contig093, |
| | | | whole genome shotgun sequence |
| | | @trn_8369 = 1 | Methylobacterium platani strain PMB02 contig093, |
| | | | whole genome shotgun sequence |
| +1 | +5 | @trn_8369 = 1 | Methylobacterium extorquens CM4, complete genome |
| | | @trn_8369 = 1 | Methylobacterium extorquens CM4, complete genome |
| | | @trn_8369 = 1 | Methylobacterium extorquens CM4, complete genome |
| | | @trn_8369 = 1 | Methylobacterium extorquens CM4, complete genome |
| | | @trn_8369 = 1 | Methylobacterium extorquens CM4, complete genome |
| +1 | +5 | @trn_8369 = 1 | Methylobacterium populi BJ001, complete genome |
| | | @trn_8369 = 1 | Methylobacterium populi BJ001, complete genome |
| | | @trn_8369 = 1 | Methylobacterium populi BJ001, complete genome |
| | | @trn_8369 = 1 | Methylobacterium populi BJ001, complete genome |
| | | @trn_8369 = 1 | Methylobacterium populi BJ001, complete genome |
| +1 | +7 | @trn_8369 = 1 | Methylobacterium nodulans ORS 2060, complete genome |
| | | @trn_8369 = 1 | Methylobacterium nodulans ORS 2060, complete genome |
| | | @trn_8369 = 1 | Methylobacterium nodulans ORS 2060, complete genome |
| | | @trn_8369 = 1 | Methylobacterium nodulans ORS 2060, complete genome |
| | | @trn_8369 = 1 | Methylobacterium nodulans ORS 2060, complete genome |
| | | @trn_8369 = 1 | Methylobacterium nodulans ORS 2060, complete genome |
| | | @trn_8369 = 1 | Methylobacterium nodulans ORS 2060, complete genome |
| +1 | +1 | @trn_8369 = 1 | Methylobacterium sp. MB200 Scaffold10_1, |
| | | | whole genome shotgun sequence |
| +1 | +2 | @trn_8369 = 1 | Rhodopseudomonas palustris DX-1, complete genome |
| | | @trn_8369 = 1 | Rhodopseudomonas palustris DX-1, complete genome |
| +1 | +5 | @trn_8369 = 1 | Methylobacterium extorquens DM4 str. DM4 chromosome, |
| | | | complete genome |
| | | @trn_8369 = 1 | Methylobacterium extorquens DM4 str. DM4 chromosome, |
| | | | complete genome |
| | | @trn_8369 = 1 | Methylobacterium extorquens DM4 str. DM4 chromosome, |
| | | | complete genome |
| | | @trn_8369 = 1 | Methylobacterium extorquens DM4 str. DM4 chromosome, |
| | | | complete genome |
| | | @trn_8369 = 1 | Methylobacterium extorquens DM4 str. DM4 chromosome, |
| | | | complete genome |
| +1 | +1 | @trn_8369 = 1 | Microvirga lotononidis strain WSM3557 |
| | | | Micloscaffold_10, whole genome shotgun sequence |
| +1 | +1 | @trn_8369 = 1 | Methylobacterium extorquens DSM 13060 ctg1157, |
| | | | whole genome shotgun sequence |
| +1 | +1 | @trn_8369 = 1 | Afipia broomeae ATCC 49717 supercont1.1, |
| | | | whole genome shotgun sequence |
| +1 | +1 | @trn_8369 = 1 | Methylobacterium mesophilicum SR1.6/6 16, |
| | | | whole genome shotgun sequence |
| +1 | +5 | @trn_8369 = 1 | Methylobacterium sp. AMS5, complete genome |
| | | @trn_8369 = 1 | Methylobacterium sp. AMS5, complete genome |
| | | @trn_8369 = 1 | Methylobacterium sp. AMS5, complete genome |
| | | @trn_8369 = 1 | Methylobacterium sp. AMS5, complete genome |
| | | @trn_8369 = 1 | Methylobacterium sp. AMS5, complete genome |
| +1 | +2 | @trn_8369 = 1 | Tardiphaga robiniae strain Vaf-07 contig_1, |
| | | | whole genome shotgun sequence |
| | | @trn_8369 = 1 | Tardiphaga robiniae strain Vaf-07 contig_1, |
| | | | whole genome shotgun sequence |
| +2 | +2 | @trn_8369 = 1 | Microvirga massiliensis strain JC119, |
| | | | whole genome shotgun sequence |
| | | @trn_8369 = 1 | Microvirga massiliensis strain JC119, |
| | | | whole genome shotgun sequence |
| +1 | +1 | @trn_8369 = 1 | Methylobacterium sp. GXF4 contig57, |
| | | | whole genome shotgun sequence |
| +1 | +5 | @trn_8369 = 1 | Methylobacterium sp. 10 K368DRAFT_scaffold00001.1, |
| | | | whole genome shotgun sequence |
| | | @trn_8369 = 1 | Methylobacterium sp. 10 K368DRAFT_scaffold00001.1, |
| | | | whole genome shotgun sequence |
| | | @trn_8369 = 1 | Methylobacterium sp. 10 K368DRAFT_scaffold00001.1, |
| | | | whole genome shotgun sequence |
| | | @trn_8369 = 1 | Methylobacterium sp. 10 K368DRAFT_scaffold00001.1, |
| | | | whole genome shotgun sequence |
| | | @trn_8369 = 1 | Methylobacterium sp. 10 K368DRAFT_scaffold00001.1, |
| | | | whole genome shotgun sequence |
| +1 | +5 | @trn_8369 = 1 | Methylobacterium sp. 77 scaffold1, whole genome shotgun sequence |
| | | @trn_8369 = 1 | Methylobacterium sp. 77 scaffold1, whole genome shotgun sequence |
| | | @trn_8369 = 1 | Methylobacterium sp. 77 scaffold1, whole genome shotgun sequence |
| | | @trn_8369 = 1 | Methylobacterium sp. 77 scaffold1, whole genome shotgun sequence |
| +1 | +1 | @trn_8369 = 1 | Methylobacterium sp. 285MFTsu5.1 H288DRAFT_scaffold00082.82, |
| | | | whole genome shotgun sequence |
| +1 | +1 | @trn_8369 = 1 | Afipia birgiae 34632 , whole genome shotgun sequence |
| +1 | +1 | @trn_8369 = 1 | Microvirga vignae strain BR3299 T20BR3299_1_paired_contig_82, |
| | | | whole genome shotgun sequence |
| +1 | +1 | @trn_8369 = 1 | Afipia sp. OHSU_II-uncloned OHSU_II_uncloned_contig_B, |
| | | | whole genome shotgun sequence |
| +1 | +1 | @trn_8369 = 1 | Methylobacterium platani JCM 14648 contig_35, |
| | | | whole genome shotgun sequence |
| +1 | +1 | @trn_8369 = 1 | Afipia sp., OHSU_II-C1 OHSU_II_C1_contig_6, |
| | | | whole genome shotgun sequence |
| +1 | +1 | @trn_8369 = 1 | Afipia sp. OHSU_II-C2 OHSU_II_C2_contig_12, |
| | | | whole genome shotgun sequence |
| +1 | +5 | @trn_8369 = 1 | Afipia sp. OHSU I-uncloned OHSU_I_uncloned_contig_3, |
| | | | whole genome shotgun sequence |
| +1 | +1 | @trn_8369 = 1 | Afipia sp. OHSU_I-C4 OHSU_I_C4_contig_10, |
| | | | whole genome shotgun sequence |
| +1 | +1 | @trn_8369 = 1 | Afipia sp. OHSU_I_C-6 OHSU_I_C6_contig_29 , |
| | | | whole genome shotgun sequence |
| +1 | +1 | @trn_8369 = 1 | Afipia sp. NBIMC_P1-C1 NBIMC_P1-C1_congit_4, |
| | | | whole genome shotgun sequence |
| +1 | +1 | @trn_8369 = 1 | Afipia sp. NBIMC_P1-C2 NBIMC_P1_C2_contig_11, |
| | | | whole genome shotgun sequence |
| +1 | +1 | @trn_8369 = 1 | Afipia sp. NBIMC_P1-C3 NBIMC_P1_C3_contig_4, |
| | | | whole genome shotgun sequence |
| +1 | +1 | @trn_8369 = 1 | Microvirga flocculans ATCC BAA-817 |
| | | | L879DRAFT_scaffold00026.26_C, |
| | | | whole genome shotgun sequence |
| +1 | +1 | @trn_8369 = 1 | Bradyrhizobium sp. URHD0069 N554DRAFT_scaffold00039.39_C, |
| | | | whole genome shotgun sequence |
| +1 | +1 | @trn_8369 = 1 | Rhizobiales bacterium YIM 77505 |
| | | | EI5 8DRAFT_untig_0_quiver_dupTri_9678 |
| | | | 0.1 C, whole genome shotgun sequence |
| +1 | +1 | @trn_8369 = 1 | Lactobacillus acidophilus CFH contig_151, |
| | | | whole genome shotgun sequence |
| +1 | +4 | @trn_8369 = 1 | Methylobacterium sp. C1, complete genome |
| | | @trn_8369 = 1 | Methylobacterium sp. C1, complete genome |
| | | @trn 8369 = 1 | Methylobacterium sp. C1, complete genome |
| | | @trn_8369 = 1 | Methylobacterium sp. C1, complete genome |
| +1 | +1 | @trn_8369 = 1 | Rhodopseudomonas sp. AAP120 AAP120_Contigs_108, |
| | | | whole genome shotgun sequence |
| +1 | +1 | @trn_8369 = 1 | Methylobacterium sp. ARG-1 Contig20, |
| | | | whole genome shotgun sequence |
| +1 | +1 | @trn_8369 = 1 | Methylobacterium sp. GXS13 contigs88, |
| | | | whole genome shotgun sequence |
| +1 | +1 | @trn_8369 = 1 | Methylobacterium sp. Leaf86 contig_36, |
| | | | whole genome shotgun sequence |
| +1 | +1 | @trn_8369 = 1 | Methylobacterium sp. Leaf87 contig_1, |
| | | | whole genome shotgun sequence |
| +1 | +1 | @trn_8369 = 1 | Methylobacterium sp. Leaf88 contig_45, |
| | | | whole genome shotgun sequence |
| +1 | +1 | @trn_8369 = 1 | Methylobacterium sp. Leaf89 contig_28, |
| | | | whole genome shotgun sequence |
| +1 | +1 | @trn_8369 = 1 | Methylobacterium sp. Leaf90 contig_11, |
| | | | whole genome shotgun sequence |
| +1 | +1 | @trn_8369 = 1 | Methylobacterium sp. Leaf91 contig_9, |
| | | | whole genome shotgun sequence |
| +1 | +1 | @trn_8369 = 1 | Methylobacterium sp. Leaf92 contig_41, |
| | | | whole genome shotgun sequence |
| +1 | +1 | @trn_8369 = 1 | Methylobacterium sp. Leaf94 contig_3, |
| | | | whole genome shotgun sequence |
| +1 | +1 | @trn_8369 = 1 | Methylobacterium sp. Leaf99 contig_1, |
| | | | whole genome shotgun sequence |
| +1 | +1 | @trn_8369 = 1 | Methylobacterium sp. Leaf100 contig_2, |
| | | | whole genome shotgun sequence |
| +1 | +1 | @trn_8369 = 1 | Methylobacterium sp. Leaf102 contig_4, |
| | | | whole genome shotgun sequence |
| +1 | +1 | @trn_8369 = 1 | Methylobacterium sp. Leaf104 contig_5, |
| | | | whole genome shotgun sequence |
| +1 | +1 | @trn_8369 = 1 | Methylobacterium sp. Leaf108 contig_2, |
| | | | whole genome shotgun sequence |
| +1 | +1 | @trn_8369 = 1 | Methylobacterium sp. Leaf111 contig_1, |
| | | | whole genome shotgun sequence |
| +1 | +1 | @trn_8369 = 1 | Methylobacterium sp. Leaf112 contig_2, |
| | | | whole genome shotgun sequence |
| +1 | +1 | @trn_8369 = 1 | Methylobacterium sp. Leaf113 contig_5, |
| | | | whole genome shotgun sequence |
| +1 | +1 | @trn_8369 = 1 | Methylobacterium sp. Leaf117 contig_5, |
| | | | whole genome shotgun sequence |
| +1 | +1 | @trn_8369 = 1 | Methylobacterium sp. Leaf119 contig_21, |
| | | | whole genome shotgun sequence |
| +1 | +1 | @trn_8369 = 1 | Methylobacterium sp. Leaf121 contig_25, |
| | | | whole genome shotgun sequence |
| +1 | +1 | @trn_8369 = 1 | Methylobacterium sp. Leaf122 contig_1, |
| | | | whole genome shotgun sequence |
| +1 | +1 | @trn_8369 = 1 | Methylobacterium sp. Leaf123 contig_4, |
| | | | whole genome shotgun sequence |
| +1 | +1 | @trn_8369 = 1 | Methylobacterium sp. Leaf125 contig_3, |
| | | | whole genome shotgun sequence |
| +1 | +1 | @trn_8369 = 1 | Rhodococcus sp. Leaf225 contig_10, |
| | | | whole genome shotgun sequence |
| +1 | +1 | @trn_8369 = 1 | Methylobacterium sp. Leaf361 contig_8, |
| | | | whole genome shotgun sequence |
| +1 | +1 | @trn_8369 = 1 | Methylobacterium sp. Leaf399 contig_2, |
| | | | whole genome shotgun sequence |
| +1 | +1 | @trn_8369 = 1 | Methylobacterium sp. Leaf456 contig_6, |
| | | | whole genome shotgun sequence |
| +1 | +1 | @trn_8369 = 1 | Methylobacterium sp. Leaf456 contig_6, |
| | | | whole genome shotgun sequence |
| +1 | +1 | @trn_8369 = 1 | Methylobacterium sp. Leaf466 contig_4, |
| | | | whole genome shotgun sequence |
| +1 | +1 | @trn_8369 = 1 | Methylobacterium sp. Leaf469 contig_2, |
| | | | whole genome shotgun sequence |
| +1 | +1 | @trn_8369 = 1 | Afipia sp. Root123D2 contig_3, |
| | | | whole genome shotgun sequence |
| +1 | +1 | @trn_8369 = 1 | Bradyrhizobium sp. DDH4-A6 CCH4-A6_contig123, |
| | | | whole genome shotgun sequence |
| +1 | +1 | @trn_8369 = 1 | Bradyrhizobium sp. CCH10-C7 CCH10-C7_contig75, |
| | | | whole genome shotgun sequence |
| +1 | +1 | @trn_8369 = 1 | Methylobacterium sp. CCH5-D2 CCH5-D2_contig721, |
| | | | whole genome shotgun sequence |
| +1 | +5 | @trn_8369 = 1 | Methylobacterium zatmanii strain PSBB041, complete genome |
| | | @trn_8369 = 1 | Methylobacterium zatmanii strain PSBB041, complete genome |
| | | @trn_8369 = 1 | Methylobacterium zatmanii strain PSBB041, complete genome |
| | | @trn_8369 = 1 | Methylobacterium zatmanii strain PSBB041, complete genome |
| | | @trn_8369 = 1 | Methylobacterium zatmanii strain PSBB041, complete genome |
| +1 | +2 | @trn_10063 = 2 | Actinophytocola xinjiangensis strain CGMCC 4.4663 contig54, |
| | | | whole genome shotgun sequence |
| +1 | +2 | @trn_10063 = 2 | Enterococcus mundtii strain SL-16 scaffold109, |
| | | | whole genome shotgun sequence |
| +1 | +2 | @trn_10063 = 2 | Escherichia coli strain 31111 31111_contig_161, |
| | | | whole genome shotgun sequence |
| +1 | +2 | @trn_10063 = 2 | Enterobacteria phage T4, complete genome |
| +1 | +2 | @trn_10063 = 2 | Salmonella enterica subsp. Enterica serovar Heidelberg |
| | | | strain NCTR-SF826 NODE_119_length 12379_cov_8.01942, |
| | | | whole genome shotgun sequence |
| +1 | +2 | @trn_10063 = 2 | Salmonella enterica subsp. Enterica serovar Dublin |
| | | | strain NCTR-SF853 NODE_1_length_169031_cov_5.39682, |
| | | | whole genome shotgun sequence |
| +1 | +2 | @trn_10063 = 2 | Escherichia phage slur14, complete genome |
| +1 | +2 | @trn_10063 = 2 | Bacteriophage RB32, complete genome |

TABLE 9

| No. | Blast | No. | | | Name | | | Taxon | | | Taxon |
|---|---|---|---|---|---|---|---|---|---|---|---|
| Reads | lines | BP | Entropy | Score | Probability | Leaves | Taxon | Code | Rank | Taxon | Code |
| 43 | 8 | 325 | 1 | 2655250 | 95.59/95.59 | 8 | Enterovirus A | 138948 | species | Enterovirus A | Enterovirus A |
| 18 | 7 | 859 | 1 | 4483980 | 158.08/145.75 | 7 | Bovine | 11128 | No rank | Bovine | Bovine |
| | | | | | | | coronavirus | | | coronavirus | coronavirus |

| | | Taxon | | | Taxon | | | Taxon |
|---|---|---|---|---|---|---|---|---|
| Tier | Taxon | Code | Tier | Taxon | Code | Tier | Taxon | Code |
| SPECIES (7) | | | GENUS (6) | | | FAMILY (5) | | |
| species | Enterovirus | 12059 | genus | Picornaviridae | 12058 | family | Picomavirales | 464095 |
| No rank | Betacorona- | 694003 | Species | Betacorona- | 694002 | genus | Coronavirinae | 693995 |
| | virus 1 | | | virus | | | | |

| | | |
|---|---|---|
| No Rank (9) | SPECIES (8) | GENUS (7) |

| | | Taxon | | | Taxon | | | Taxon |
|---|---|---|---|---|---|---|---|---|
| Tier | Taxon | Code | Tier | Taxon | Code | Tier | Taxon | Code |
| ORDER (4) | | | NO RANK (3) | | | NO RANK (2) | | |
| order | ssRNA | 35278 | no rank | ssRNA | 439488 | no rank | Viruses | 10239 |
| | positive-strand | | | viruses | | | | |
| | viruses' | | | | | | | |
| | no DNA stage | | | | | | | |
| sub- | Coronaviridae | 11118 | family | Nidovirales | 76804 | order | | 35278 |
| family | | | | | | | | |

| | | |
|---|---|---|
| SUBFAMILY (6) | FAMILY (5) | ORDER (4) |

| | | Taxon | | | Taxon | | | Taxon | | | Taxon |
|---|---|---|---|---|---|---|---|---|---|---|---|
| Tier | Taxon | Code | Tier | Taxon | Code | Tier | Taxon | Code | Tier | Taxon | Code |
| SUPER KINGDOM (1) | | | ROOT (0) | | | | | | | | |
| super | — | — | — | — | — | | | | | | |
| kingdom | | | | | | | | | | | |
| no rank | ssRNA | 439488 | no rank | Viruses | 10239 | super | — | — | — | — | — |
| | viruses | | | | | kingdom | | | | | |

| | | | |
|---|---|---|---|
| NO RANK (3) | NO RANK (2) | SUPER KINGDOM (1) | ROOT (0) |

TABLE 10

| | Taxon | | Read | Tier | Tier | Branches |
|---|---|---|---|---|---|---|
| Name | ID | Tier | No. | N | Probability | in Tier |
| root | 1 | Root | 19 | 0 | 100.0/100.0 | 19 |
| Viruses | 10239 | Superkingdom | 8 | 1 | 184.42/169.61 | 8 |
| ssRNA viruses | 439488 | No rank | 7 | 2 | 208.29/191.53 | 7 |
| ssRNA positive-strand viruses' no DNA stage | 35278 | No rank | 7 | 3 | 208.29/191.53 | 7 |
| Nidovirales | 76804 | Order | 7 | 4 | 208.29/191.53 | 7 |
| Coronaviridae | 11118 | Family | 7 | 5 | 208.29/191.53 | 7 |
| Coronavirinae | 693995 | Subfamily | 7 | 6 | 208.29/191.53 | 7 |
| Betacoronavirus | 694002 | Genus | 6 | 7 | 158.84/146.81 | 6 |
| Betacoronavirus 1 | 694003 | Species | 6 | 8 | 158.84/146.81 | 6 |
| Bovine coronavirus | 11128 | No rank | 6 | 9 | 158.84/146.81 | 6 |
| Cellular organism | 131567 | No rank | 12 | 1 | 715385.26/694252.0 | 12 |
| Bacteria | 2 | Superkingdom | 12 | 2 | 715385.26/694252.0 | 12 |
| Proteobacteria | 1224 | Phylum | 12 | 3 | 715385.26/694252.0 | 12 |
| Alphaproteobacteria | 28211 | Class | 3 | 4 | 7692.73/7330.28 | 3 |
| Rhizobiales | 356 | Order | 3 | 5 | 7692.73/7330.28 | 3 |
| Methylobacteriaceae | 119045 | Family | 3 | 6 | 6073.02/5786.89 | 3 |
| Methylobacterium | 407 | Genus | 3 | 7 | 5666.98/5399.97 | 3 |
| Methylobacterium sp. Leaf466 | 1736386 | Species | 1 | 8 | 5.79/5.52 | 1 |
| Methylobacterium sp. Leaf399 | 1736364 | Species | 1 | 8 | 5.79/5.52 | 1 |
| Methylobacterium sp. Leaf108 | 1736256 | Species | 1 | 8 | 5.79/5.52 | 1 |
| Terrabacteria group | 1783272 | No rank | 3 | 3 | 17.61/16.75 | 3 |
| Actinobacteria | 201174 | Phylum | 3 | 4 | 11.9/11.32 | 3 |
| Actinobacteria | 1760 | Class | 3 | 5 | 11.9/11.32 | 3 |
| Streptomycetales | 85011 | Order | 2 | 6 | 9.11/8.68 | 2 |
| Streptomycetaceae | 2062 | Family | 2 | 7 | 9.11/8.68 | 2 |
| Streptomyces | 1183 | Genus | 2 | 8 | 9.11/8.668 | 2 |
| Streptomyces purpurogeneiscleroticus | 68259 | Species | 2 | 9 | 9.11/8.68 | 2 |
| Methylobacterium phyllosphaerae | 418223 | Species | 3 | 8 | 135.66/129.26 | 3 |
| Methylobacterium sp. B1 | 91459 | Species | 2 | 8 | 29.86/28.45 | 2 |
| Methylobacterium populi | 223967 | Species | 1 | 8 | 17.72/16.9 | 1 |
| Methylobacterium sp. Leaf361 | 1736352 | Species | 1 | 8 | 13.98/13.2 | 1 |
| Methylobacterium radiotolerans | 31998 | Species | 1 | 8 | 23.48/22.39 | 1 |
| Methylobacterium extorquens group | 57882 | Species group | 1 | 8 | 284/92/271.63 | 1 |
| Methylobacterium extorquens | 408 | Species | 1 | 8 | 284.92/271.63 | 1 |
| Methylobacterium sp. C1 | 1479019 | Species | 1 | 8 | 8.6/8.2 | 1 |
| Methylobacterium sp. AMS5 | 925818 | Species | 1 | 8 | 12.76/12.17 | 1 |
| Methylobacterium extorquens DM4 | 661410 | No rank | 1 | 10 | 12.76/12.17 | 1 |
| Methylobacterium extorquens AM1 | 272630 | No rank | 1 | 10 | 12.76/12.17 | 1 |
| Methylobacterium extorquens CM4 | 440085 | No rank | 1 | 10 | 12.76/12.17 | 1 |
| Methylobacterium populi BJ001 | 441620 | No rank | 1 | 9 | 12.76/12.17 | 1 |
| Methylobacterium radiotolerans JCM 2831 | 426355 | No rank | 1 | 9 | 17.72/16.9 | 1 |
| Methylobacterium extorquens PA1 | 419610 | No rank | 1 | 10 | 12.76/12.17 | 1 |
| Methylobacterium aquaticum | 270351 | Species | 1 | 8 | 76.17/72.62 | 1 |
| Methylobacterium platani | 427683 | Species | 1 | 8 | 7.7/7.34 | 1 |
| Methylobacterium sp. WSM2598 | 398261 | Species | 1 | 8 | 15.73/14.99 | 1 |
| Methylobacterium sp. 4-46 | 426117 | Species | 1 | 8 | 15.73/15.0 | 1 |
| Methylobacterium nodulans | 114616 | Species | 1 | 8 | 19.27/18.37 | 1 |
| Methylobacterium nodulans ORS 2060 | 460265 | No rank | 1 | 9 | 19.27/18.37 | 1 |
| Microvirga | 186650 | Genus | 1 | 7 | 10.23/9.76 | 1 |
| Brandyrhizobiaceae | 41294 | Family | 1 | 6 | 217.58/207.43 | 1 |
| Rhodopseudomonas | 1073 | Genus | 1 | 7 | 20 94/19 96 | 1 |
| Rhodopseudomonas palustris | 1076 | Species | 1 | 8 | 16.52/15.75 | 1 |
| Methylobacterium sp. 10 | 1101191 | Species | 1 | 8 | 10/23/9.76 | 1 |
| Methylobacterium sp. 77 | 1101192 | Species | 1 | 8 | 6.97/6.64 | 1 |
| Afipia | 1033 | Genus | 1 | 7 | 50.21/47.87 | 1 |
| Firmicutes | 1239 | Phylum | 1 | 4 | 36.18/33.28 | 1 |
| Bacilli | 91061 | Class | 1 | 5 | 36.18/33.28 | 1 |
| Lactobacillales | 186826 | Order | 1 | 6 | 36.18/33.28 | 1 |
| Pseudonocardiales | 85010 | Order | 1 | 6 | 36.18/33.28 | 1 |
| Pseudonocardiaceae | 2070 | Family | 1 | 7 | 36.18/33.28 | 1 |
| Actinophytocola | 695999 | Genus | 1 | 8 | 36.18/33.28 | 1 |
| Actinophytocola xinjiangensis | 485062 | Species | 1 | 9 | 36.18/33.28 | 1 |
| Enterococcaceae | 81852 | Family | 1 | 7 | 36.18/33.28 | 1 |
| Enterococcus | 1350 | Genus | 1 | 8 | 36.18/33.28 | 1 |
| Enterococcus mundtii | 53346 | Species | 1 | 9 | 36.18/33.28 | 1 |
| Gammaproteobacteria | 1236 | Class | 6 | 4 | 789092.3/767218.38 | 6 |
| Enterobacterales | 91347 | Order | 6 | 5 | 787722.01/765886.08 | 6 |
| Enterobacteriaceae | 543 | Family | 6 | 6 | 783632.28/761909.72 | 6 |
| Escherichia | 561 | Genus | 6 | 7 | 441052.61/428826.48 | 6 |
| Escherichia coli | 562 | Species | 6 | 8 | 429805.73/417891.36 | 6 |
| dsDNA viruses/no RNA stage | 35237 | No rank | 1 | 2 | 168.9/155.35 | 1 |
| Caudovirales | 28883 | Order | 1 | 3 | 168.9/155.35 | 1 |
| Myoviridae | 10662 | Family | 1 | 4 | 168.9/155.35 | 1 |
| Tevenvirinae | 1998136 | Subfamily | 1 | 5 | 168.9/155.35 | 1 |
| T4virus | 10663 | Genus | 1 | 6 | 168.9/155.35 | 1 |
| Enterobacteria phage T4 sensu lato | 348604 | Species | 1 | 7 | 36.18/33.28 | 1 |
| Enterobacteria phage T4 | 10665 | No rank | 1 | 8 | 36.18/33.28 | 1 |
| Salmonella | 590 | Genus | 4 | 7 | 1323.2/1292.13 | 4 |
| Salmonella enterica | 28901 | Species | 4 | 8 | 1323.2/1292.13 | 4 |
| Salmonella enterica subsp. enterica | 59201 | Subspecies | 4 | 9 | 1186.63/1158.77 | 4 |
| Salmonella enterica subsp. Serovar Heidelberg | 611 | No rank | 1 | 10 | 31.28/2877 | 1 |
| Salmonella enterica subsp. Enterica serovar Dublin | 98360 | No rank | 1 | 10 | 31.28/28.77 | 1 |
| Unclassified T4virus | 329380 | No rank | 1 | 7 | 82.03/75.45 | 1 |
| Escherichia phage slur08 | 1720501 | Species | 1 | 8 | 31.28/28.77 | 1 |
| Escherichia phage slur14 | 1720504 | No rank | 1 | 9 | 31.28/28.77 | 1 |
| Enterobacteria phage RB32 | 45406 | Species | 1 | 8 | 25.07/23.05 | 1 |
| Salmonella enterica subsp. Enterica serovar Newport | 108619 | No rank | 1 | 10 | 52.78/50.27 | 1 |
| Salmonella enterica subsp. Enterica serovar Newport str. | 1454627 | No rank | 1 | 11 | 52.78/50.27 | 1 |
| Salmonella enterica subsp. Enterica serovar Enteritidis | 149539 | No rank | 1 | 10 | 869.04/827.66 | 1 |
| Salmonella enterica subsp. Enterica serovar Typhimurium | 90371 | No rank | 2 | 10 | 498.01/491.59 | 2 |
| Betaproteobacteria | 28216 | Class | 3 | 4 | 5165.35/5001.35 | 3 |
| Burkholderiales | 80840 | Order | 3 | 5 | 5165.35/5001.35 | 3 |
| Unclassified Burkholderiales | 119065 | No rank | 1 | 6 | 329.44/304.83 | 1 |
| Burkholderiales Genera incertae sedis | 224471 | No rank | 1 | 7 | 329.44/304.83 | 1 |
| Aquabacterium | 92793 | Genus | 1 | 8 | 329.44/304.83 | 1 |
| Aquabacterium sp. NJ1 | 1538295 | Species | 1 | 9 | 329.44/304.83 | 1 |
| Escherichia coli O157:H7 | 83334 | No rank | 1 | 9 | 288.77/279.12 | 1 |
| Shigella | 620 | Genus | 2 | 7 | 10518.15/10338.57 | 2 |
| Escherichia coli K-12 | 83333 | No rank | 2 | 9 | 295.75/290.7 | 2 |
| Shigella flexneri | 623 | Species | 1 | 8 | 8.69/8.37 | 1 |
| Escherichia coli O104:H4 | 1038927 | No rank | 2 | 9 | 1190.2/1150.41 | 2 |
| Shigella sonnei | 624 | Species | 1 | 8 | 17651.57/17619.45 | 1 |
| Escherichia coli O45:H2 | 1078032 | No rank | 1 | 9 | 8.69/83.7 | 1 |
| Escherichia coli O104:H4 str. C227-11 | 1048254 | No rank | 1 | 10 | 8.69/83.7 | 1 |
| Escherichia coli O157 | 104010 | No rank | 1 | 9 | 8.69/83.7 | 1 |
| Escherichia coli str. K-12 substr. MG1655 | 51145 | No rank | 1 | 10 | 19.26/18.53 | 1 |
| Escherichia coli B | 37762 | No rank | 1 | 9 | 8.69/8.37 | 1 |
| Klebsiella | 570 | Genus | 1 | 7 | 64852.37/64734.34 | 1 |
| Klebsiella Pneumoniae | 573 | Species | 1 | 8 | 60077.77/59968.43 | 1 |
| Enterobacter | 547 | Genus | 1 | 7 | 1972.55/1968.96 | 1 |
| Enterobacter clocacae complex | 352476 | Species Group | 1 | 8 | 1972.55/198.96 | 1 |
| Enterobacter cloacae | 550 | Species | 1 | 9 | 204.77/204.4 | 1 |
| Salmonella enterica subsp. Enterica serovar Agona | 58095 | No rank | 1 | 10 | 10.36/10.34 | 1 |
| Klebsiella michiganesis | 1134687 | Species | 1 | 8 | 91.92/91.75 | 1 |
| Citrobacter | 544 | Genus | 1 | 7 | 252.02/251.56 | 1 |
| Citrobacter amalonaticus | 35703 | Species | 1 | 8 | 23.14/23.1 | 1 |
| Escherichia fergusonii | 564 | Species | 1 | 8 | 307.97/307.41 | 1 |
| Salmonella enterica subsp. Enterica serovar Berta | 28142 | No rank | 1 | 10 | 10.36/10.34 | 1 |
| Salmonella enterica subsp. Enterica serovar Berta | 1242696 | No rank | 1 | 11 | 10.36/10.34 | 1 |
| str. SA20103550 | | | | | | |
| Yersiniaceae | 1903411 | Family | 1 | 6 | 7.91/7.9 | 1 |
| Serratia | 613 | Genus | 1 | 7 | 7.91/7.9 | 1 |
| Serratia marcescens | 615 | Species | 1 | 8 | 7.91/7.9 | 1 |
| Enterobacter sp. BIDMC99 | 1686398 | Species | 1 | 9 | 124.38/124.15 | 1 |
| Enterobacter sp. BWH63 | 1686397 | Species | 1 | 9 | 63.27/63.16 | 1 |
| Citrobacter freundii complex | 1334959 | No rank | 1 | 8 | 123.17/122.95 | 1 |
| Citrobacter sp. MGH103 | 1686378 | Species | 1 | 9 | 62.63/62.51 | 1 |
| Burkholderiaceae | 119060 | Family | 2 | 6 | 5858.45/5707.61 | 2 |
| Burkholderia | 32008 | Genus | 2 | 7 | 743.83/724.68 | 2 |
| Burkholderia sp. K24 | 1472716 | Species | 2 | 8 | 743.83/724.68 | 2 |
| Paraburkholderia | 1822464 | Genus | 2 | 7 | 2531.78/2466.59 | 2 |
| Paraburkholderia fungorum | 134537 | Species | 2 | 8 | 2531.78/2466.59 | 2 |
| Paraburkholderia fungorum NBRC 102489 | 1218077 | No rank | 2 | 9 | 743.82/724.68 | 2 |
| Alphacoronavirus | 693996 | Genus | 1 | 7 | 341.44309.7 | 1 |
| Human coronavirus 229E | 11137 | Species | 1 | 8 | 341.44/309.7 | 1 |
| Methylobacterium sp. UNCCL110 | 1449057 | Species | 1 | 8 | 70.65/55.71 | 1 |

What is claimed is:

1. A computer-implemented method for identifying pathogens in a sample comprising a plurality of genetic sequences, the method comprising:
receiving a plurality of electronic sequence reads corresponding to the plurality of genetic sequences of the sample;
electronically sampling a set of electronic sequence reads from the plurality of electronic sequence reads;
iteratively and electronically comparing the sampled set against a plurality of pathogen sequences to create a detection group;
electronically populating a putative genome data structure with the detection group; and
electronically comparing the sample set against the putative genome data structure to:
measure a distance score between each electronic sequence read of the sampled set to each pathogen sequence of the putative genome data structure;
calculate a hit score from the respective distance scores for each electronic sequence read of the sampled set, wherein the hit score is a comparison of the distance score of a respective electronic sequence read to a threshold value;
form a plurality of clusters of the electronic sequence reads of the sample set such that a hit score of the cluster is maximized while a difference in distance scores within the cluster is minimized; and
display a respective taxonomic group assigned to electronic sequence reads of the sample set based on the plurality of clusters.

2. The method of claim 1, wherein electronically comparing the electronic sequence reads of the sample set against the putative genomic data structure further comprises:
electronically calculating an entropy score for each electronic sequence read of the sample set, wherein the entropy score is the hit score per taxon level.

3. The method of claim 2, wherein a calculated entropy score of 1 indicates a direct match of the respective electronic sequence read to one pathogen sequence of the putative genomic data structure.

4. The method of claim 1, further comprising:
electronically reverse mapping the plurality of electronic sequence reads against a filtered plurality of known genetic sequences prior to electronically sampling.

5. The method of claim 4, wherein the filtered plurality of known genetic sequences comprises human genome sequences, taxonomic information, or both.

6. The method of claim 1, wherein the plurality of pathogen sequences comprises genomes of known pathogens of concern.

7. The method of claim 1, wherein the respective taxonomic group assigned to the electronic sequence reads of the sample set is selected from the group consisting of known pathogens and unknown pathogens.

8. The method of claim 1, wherein each electronic sequence read of the plurality is characterized by a respective length of at least 75 base pairs.

9. The method of claim 1, wherein electronic sequence reads of the plurality that cannot be compared to any pathogen sequence of the plurality may include a protein sequence, a motif sequence, a toxin-virulent sequence, or a warfare sequence.
